(19) United States
(12) Patent Application Publication
(10) Pub. No.: US 2019/0314379 A1
(43) Pub. Date: Oct. 17, 2019

(54) METHODS OF USING CANNABINOID CB2 RECEPTOR AGONIST COMPOSITIONS TO SUPPRESS AND PREVENT OPIOID TOLERANCE AND WITHDRAWAL IN A SUBJECT

(71) Applicant: Indiana University Research and Technology Corporation, Indianapolis, IN (US)

(72) Inventors: Andrea Grace HOHMANN, Bloomington, IN (US); Ken Paul MACKIE, Bloomington, IN (US); Xiaoyan LIN, Bloomington, IN (US); Amey S. DHOPESHWARKAR, Bloomington, IN (US)

(73) Assignee: Indiana University Research and Technology Corporation, Indianapolis, IN (US)

(21) Appl. No.: 16/256,787

(22) Filed: Jan. 24, 2019

Related U.S. Application Data
(60) Provisional application No. 62/621,363, filed on Jan. 24, 2018.

(51) Int. Cl. A61K 31/52; A61K 31/366; A61K 31/485; A61P 25/04; A61P 25/36

(52) U.S. Cl. CPC A61K 31/52; A61K 31/366; A61P 25/36; A61P 25/04; A61K 31/485

(57) ABSTRACT

The present disclosure relates to methods of using cannabinoid CB₂ receptor agonist compositions to suppress pain (e.g., neuropathic pain), opioid tolerance, and/or opioid-induced physical dependence in a subject.

### CROSS-REFERENCE TO RELATED APPLICATION

This application claims the benefit under 35 USC § 119(e) of U.S. Provisional Patent Application Ser. No. 62/621,363, filed on Jan. 24, 2018, the entire disclosure of which is incorporated herein by reference.

### GOVERNMENT LICENSE RIGHTS

This invention was made with government support under DA041229 awarded by the National Institutes of Health. The government has certain rights in the invention.

### FIELD OF THE PRESENT DISCLOSURE

The present disclosure relates to methods of using cannabinoid CB₂ receptor agonist compositions, including a slow signaling G-protein biased CB2 agonist, to suppress pain (e.g., neuropathic pain), opioid tolerance, and/or opioid-induced physical dependence in a subject.

### BACKGROUND

Morphine suppresses many types of pain, but tolerance, physical dependence, and unwanted side effects limit its clinical use. Identification of therapeutic strategies for blocking opioid tolerance and dependence when treating subjects with pain has therefore evolved as an area of intense research interest. Adjunctive pharmacotherapies that combine mechanistically distinct analgesics represent one such approach.

For example, opioid and cannabinoid CB₁ G protein-coupled receptors are often coexpressed in the central nervous system (CNS). Opioid and cannabinoid CB₁ receptors can functionally interact by receptor heterodimerization or signaling cross-talk. Although activation of both opioid and cannabinoid CB₁ G protein-coupled receptors produces analgesia, undesirable pharmacologic effects limit their clinical use.

In contrast, CB₂ receptors are primarily expressed on immune cells but may be induced in the CNS in response to injury. Activation of cannabinoid CB₂ receptors produces antinociceptive efficacy in many preclinical pain models without the unwanted side effects associated with CNS CB₁ receptor activation. Cannabinoid CB₂ receptors have also been implicated in facilitating morphine antinociception in normal and inflammatory pain conditions.

However, whether cannabinoid CB₂ receptor agonists suppress morphine tolerance or dependence in other pain models, such as neuropathic pain models, is currently unknown. Therefore, an approach presented in the present disclosure aims at harnessing the therapeutic potential of cannabinoid CB₂ receptor agonists to suppress pain without producing CB₁-mediated cannabimimetic effects. More specifically, the present disclosure is directed to a cannabinoid CB₂ receptor agonist composition and methods of using the cannabinoid CB₂ receptor agonist composition to treat subjects having neuropathic pain.

### SUMMARY OF THE INVENTION

The present disclosure provides a method of suppressing neuropathic pain in a subject without producing tolerance. The present method comprises, consists essentially of, or consists of: a) administering a pharmaceutical composition comprising a cannabinoid CB2 receptor agonist compound to the subject, b) activating one or more G-protein signaling pathways that effects neuropathic pain in the subject, c) improving one or more clinical manifestations of the neuropathic pain in the subject, and d) suppressing the neuropathic pain in the subject. The subject of the present method is a human or a rodent, such as a mouse.

The cannabinoid CB₂ receptor agonist compounds of the present method of suppressing neuropathic pain may comprise, consist essentially of, or consist of a LY2828360 compound, an AM1710 compound, or an analog, a derivative, a pharmaceutically acceptable salt, a hydrate, a prodrug, or a combination thereof. The LY2828360 of the method of suppressing pain may comprise, consist essentially of, or consist of 8-(2-chlorophenyl)-2-methyl-6-(4-methylpiperazin-1-yl)-9-(tetrahydro-2H-pyran-4-yl)-9H-purine. The LY2828360 of the present disclosure may also comprise, consist essentially of, or consist of the following chemical structure:

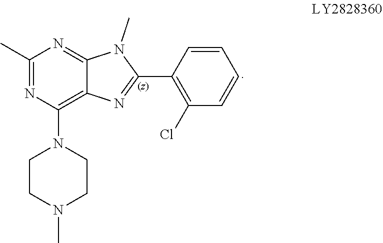 [SMILES: Cc1nc(N2CCN(C)CC2)c2nc(-c3ccccc3Cl)n(C)c2n1]

The AM1710 compound of the method of suppressing pain may comprise, consist essentially of, or consist of 3-(1,1-dimethyl-heptyl)-1-hydroxy-9-methoxy-benzo(c) chromen-6-one. The AM1710 compound of the present disclosure may also comprise, consist essentially of, or consist of the following chemical structure:

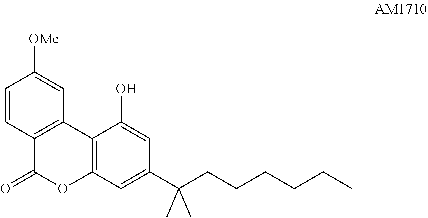 [SMILES: CCCCCCC(C)(C)c1cc(O)c2c(c1)oc(=O)c1ccc(OC)cc12]

In one embodiment, the present method of suppressing neuropathic pain may comprise activating one or more G-protein signaling pathways by the LY2828360 compound occurs through a slow signaling mechanism. In another embodiment of the present method of suppressing neuropathic pain, activating the one or more G-protein signaling pathways by the AM1710 compound occurs through a fast signaling mechanism.

In addition, the present disclosure provides a method of reducing or preventing opioid withdrawal in a subject. The method of reducing or preventing opioid withdrawal comprises, consists essentially of, or consists of: a) administering to the subject a pharmaceutical composition comprising a LY2828360 compound, an AM1710 compound, or an analog, a derivative, a pharmaceutically acceptable salt, a hydrate, a prodrug, or a combination thereof, b) activating one or more G-protein signaling pathways that effects opioid withdrawal in the subject, c) improving one or more clinical manifestations of the opioid withdrawal in the subject, and d) reducing or preventing opioid withdrawal in the subject.

The LY2828360 compound of the method of reducing or preventing opioid withdrawal may comprise, consist essentially of, or consist of (8-(2-chlorophenyl)-2-methyl-6-(4-methylpiperazin-1-yl)-9-(tetrahydro-2H-pyran-4-yl)-9H-purine). The LY2828360 of the method of reducing opioid withdrawal may also comprise, consist essentially of, or consist of the following chemical structure:

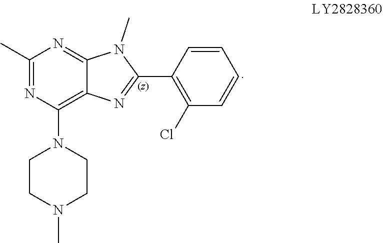 [SMILES: Cc1nc(N2CCN(C)CC2)c2nc(-c3ccccc3Cl)n(C)c2n1]

The AM1710 compound of the method of reducing or preventing opioid may comprise, consist essentially of, or consist of 3-(1,1-dimethyl-heptyl)-1-hydroxy-9-methoxy-benzo(c) chromen-6-one. The AM1710 compound of the present disclosure may also comprise, consist essentially of, or consist of the following chemical structure:

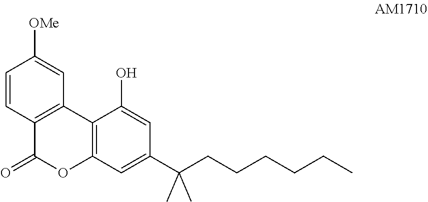 [SMILES: CCCCCCC(C)(C)c1cc(O)c2c(c1)oc(=O)c1ccc(OC)cc12]

In the present method of reducing or preventing opioid withdrawal, the one or more clinical manifestations of the opioid withdrawal may comprise, consist essentially of, or consist of a plurality of withdrawal jumps by the subject. The subject of the method of reducing or preventing opioid withdrawal may be a human or a rodent, such as a mouse.

Finally, the present disclosure is directed to a method of reducing or preventing the development of opioid tolerance in a subject. The method of reducing or preventing the development of opioid tolerance comprises, consists essentially of, or consists of: a) co-administering to the subject one or more pharmaceutical compositions comprising: 1) a LY2828360 compound, an AM1710 compound, or an analog, a derivative, a pharmaceutically acceptable salt, a hydrate, a prodrug, or a combination thereof and 2) an opioid, b) activating one or more G-protein signaling pathways that effects opioid tolerance in the subject, c) suppressing development or presentation of one or more clinical manifestations of the opioid tolerance in the subject, and d) reducing or preventing the development of opioid tolerance in the subject.

The subject of the method of reducing or preventing the development of opioid tolerance may be a human or a rodent, such as a mouse. The opioid of this method of reducing or preventing the development of opioid tolerance may be selected from the group consisting of morphine, codeine, oxycodone, oxycontin, hydrocodone, methadone, meperidine, buprenorphine, hydromorphone, tapentadol, tramadol, heroin, and fentanyl. The LY2828360 compound and the AM1710 compound of the method of reducing or preventing the development of opioid tolerance comprises 8-(2-chlorophenyl)-2-methyl-6-(4-methylpiperazin-1-yl)-9-(tetrahydro-2H-pyran-4-yl)-9H-purine and 3-(1,1-dimethyl-heptyl)-1-hydroxy-9-methoxy-benzo(c) chromen-6-one, respectively. Finally, the method of reducing or preventing the development of opioid tolerance comprises the LY2828360 compound that has the following chemical structure:

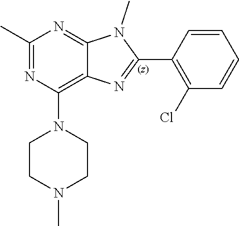 [SMILES: Cc1nc(N2CCN(C)CC2)c2nc(-c3ccccc3Cl)n(C)c2n1]

or the AM1710 compound that has the following chemical structure:

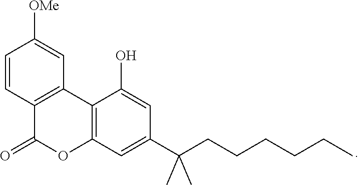 [SMILES: CCCCCCC(C)(C)c1cc(O)c2c(c1)oc(=O)c1ccc(OC)cc12]

### BRIEF DESCRIPTION OF THE DRAWINGS

A brief description of the drawings is as follows.

FIG. 1A is a chemical structure of one embodiment of the cannabinoid CB₂ receptor agonist composition, LY2828360, of the present disclosure.

FIG. 1B is a chemical structure of another embodiment of the cannabinoid CB₂ receptor agonist composition, AM1710, of the present disclosure.

FIG. 2A is a graph that shows the arrestin recruitment of compositions LY2828360 and CP55940 in CHO cells stably expressing mouse CB₂ receptors.

FIG. 2B is a graph that shows the concentration of compositions LY2828360 and CP55940 at the surface levels of HEK cells stably expressing mouse CB₂ receptors.

FIG. 2C is a graph that shows the inhibition of accumulation of forskolin-stimulated cAMP levels by LY2828360 and CP55940.

FIG. 2D is a graph that shows the inhibition of accumulation of forskolin-stimulated cAMP levels by LY2828360 and CP55940 after pertussis toxin (PTX) treatment.

FIG. 2E is a graph that shows the inhibition of accumulation of forskolin-stimulated cAMP levels by LY2828360 and CP55940 after 5 minutes.

FIG. 2F is a graph that shows the inhibition of accumulation of forskolin-stimulated cAMP levels by LY2828360 and CP55940 after 30 minutes.

FIG. 3A is a graph that shows the effect of LY2828360 and CP55940 on phosphorylated ERK1/2 levels in HEK cells stably expressing mouse CB₂ receptors.

FIG. 3B is a graph that shows the effect of LY2828360 and CP55940 on phosphorylated ERK1/2 levels after pertussis toxin (PTX) treatment.

FIG. 3C is a graph that shows the effect of LY2828360 and CP55940 on phosphorylated ERK1/2 levels after 5 minutes.

FIG. 3D is a graph that shows the effect of LY2828360 and CP55940 on phosphorylated ERK1/2 levels after 20 minutes.

FIG. 4A is a graph that shows the effect of Paclitaxel (Pac) treatment and a non-chemotherapy Cremphor (CR) vehicle control treatment on subjects that received mechanical stimulation.

FIG. 4B is a graph that shows the effect of Paclitaxel (Pac) treatment and a non-chemotherapy, control Cremphor (CR) vehicle treatment on subjects that received cold stimulation

FIG. 4C is a graph that shows the dose response of LY2828360 administered to Paclitaxel-treated and Cremphor vehicle-treated subjects that experienced mechanical allodynia.

FIG. 4D is a graph that shows the dose response of LY2828360 administered to Paclitaxel-treated and Cremphor vehicle-treated subjects that experienced cold allodynia.

FIG. 4E is a graph that shows the effect of LY2828360 administered to Paclitaxel-treated and Cremphor vehicle-treated subjects that experienced mechanical allodynia.

FIG. 4F is a graph that shows the effect of LY2828360 administered to Paclitaxel-treated and Cremphor vehicle-treated subjects that experienced cold allodynia.

FIG. 5A is a schematic that depicts one testing protocol embodiment used to evaluate the two phases of treatment (i.e., Phase I and Phase II) during the maintenance of neuropathic pain.

FIG. 5B is a graph that shows the effect of LY2828360 and Morphine administered during Phase I and Phase II of treatment, respectively, to Paclitaxel-treated wildtype subjects that experienced mechanical allodynia.

FIG. 5C is a graph that shows the effect of LY2828360 and Morphine administered during Phase I and Phase II of treatment, respectively, to Paclitaxel-treated wildtype subjects that experienced cold allodynia.

FIG. 5D is a graph that shows the effect of LY2828360 and Morphine administered during Phase I and Phase II of treatment, respectively, to Paclitaxel-treated wildtype and CB₂ Knockout (CB₂KO) subjects that experienced mechanical allodynia.

FIG. 5E is a graph that shows the effect of LY2828360 and Morphine administered during Phase I and Phase II of treatment, respectively, to Paclitaxel-treated wildtype and CB₂KO subjects that experienced cold allodynia.

FIG. 6A is a schematic that depicts another testing protocol embodiment used to evaluate the two phases of treatment (i.e., Phase I and Phase II) during the maintenance of neuropathic pain.

FIG. 6B is a graph that shows the effect of Morphine and LY2828360 administered during Phase I and Phase II of treatment, respectively, to Paclitaxel-treated wildtype subjects that experienced mechanical allodynia.

FIG. 6C is a graph that shows the effect of Morphine and LY2828360 administered during Phase I and Phase II of treatment, respectively, to Paclitaxel-treated wildtype subjects that experienced cold allodynia.

FIG. 6D is a graph that shows the effect of Morphine and LY2828360 administered during Phase I and Phase II of treatment, respectively, to Paclitaxel-treated wildtype and CB₂KO subjects that experienced mechanical allodynia.

FIG. 6E is a graph that shows the effect of Morphine and LY2828360 administered during Phase I and Phase II of treatment, respectively, to Paclitaxel-treated wildtype and CB₂KO subjects that experienced cold allodynia.

FIG. 7A is a graph that shows the effect of coadministration of Morphine and LY2828360 to Paclitaxel-treated wildtype and CB₂KO subjects that experienced mechanical allodynia.

FIG. 7B is a graph that shows the effect of coadministration of Morphine and LY2828360 to Paclitaxel-treated wildtype and CB₂KO subjects that experienced cold allodynia.

FIG. 8A is a graph that shows the effect on naloxone-precipitated opioid withdrawal of Morphine and LY2828360 administered during each of Phase I and Phase II of treatment, respectively, on Paclitaxel-treated wildtype subjects.

FIG. 8B is a graph that shows the effect on naloxone-precipitated opioid withdrawal of Morphine and LY2828360 administered during each of Phase I and Phase II of treatment, respectively, on CB₂KO subjects.

FIG. 8C is a graph that shows the effect of LY2828360 and Morphine administered during Phase I and Phase II of treatment, respectively, to Paclitaxel-treated wildtype and CB₂KO subjects.

FIG. 8D is a graph that shows the effect of coadministration of LY2828360 and Morphine treatment to Paclitaxel-treated wildtype and CB₂KO subjects.

FIG. 8E is a graph that shows the changes in body weight of Paclitaxel-treated wildtype and CB₂KO subjects treated with Morphine and/or LY2828360 after naloxone injection.

FIG. 9A is a graph that shows the effect of AM1710 administered during Phase I and Morphine administered in Phase II of treatment to Paclitaxel-treated wildtype subjects that experienced mechanical allodynia.

FIG. 9B is a graph that shows the effect of AM1710 administered during Phase I and Morphine administered in Phase II of treatment to Paclitaxel-treated wildtype subjects that experienced cold allodynia.

FIG. 10A is a graph that shows the effect on naloxone-precipitated opioid withdrawal jumps when AM1710 and/or Morphine is administered to Paclitaxel-treated wildtype subjects.

FIG. 10B is a graph that shows the changes in body weight of Paclitaxel-treated wildtype subjects treated with AM1710 and/or Morphine after naloxone injection.

FIG. 10C is a graph that shows the changes in body temperature of Paclitaxel-treated wildtype subjects treated with AM1710 and/or Morphine after naloxone injection.

FIG. 11A is a graph that shows the concentration of compositions LY2828360 and CP55940 at the surface levels of HEK cells stably expressing human CB₂ receptors.

FIG. 11B is a graph that shows the inhibition of accumulation of forskolin-stimulated cAMP levels by LY2828360 and CP55940 in HEK cells stably expressing human CB₂ receptors.

FIG. 11C is a graph that shows the inhibition of accumulation of forskolin-stimulated cAMP levels by LY2828360 and CP55940 after pertussis toxin (PTX) treatment in HEK cells stably expressing human CB₂ receptors.

FIG. 11D is a graph that shows the inhibition of accumulation of forskolin-stimulated cAMP levels by LY2828360 and CP55940 after 5 minutes in HEK cells stably expressing human CB₂ receptors.

FIG. 11E is a graph that shows the inhibition of accumulation of forskolin-stimulated cAMP levels by LY2828360, CP55940, and SR144528 after 35 minutes in HEK cells stably expressing human CB₂ receptors.

FIG. 12A is a graph that shows the effect of LY2828360 and CP55940 on phosphorylated ERK1/2 levels after 5 minutes in HEK cells stably expressing human CB₂ receptors.

FIG. 12B is a graph that shows the effect of LY2828360 and CP55940 on phosphorylated ERK1/2 levels after 30 minutes in HEK cells stably expressing human CB₂ receptors.

FIG. 12C is a graph that shows the effect of LY2828360 and CP55940 on phosphorylated ERK1/2 levels after pertussis toxin (PTX) treatment in HEK cells stably expressing human CB₂ receptors.

FIG. 12D is a graph that shows the effect of LY2828360, CP55940, and SR144528 on phosphorylated ERK1/2 levels after 30 minutes in HEK cells stably expressing human CB₂ receptors.

FIG. 13A is a graph that shows the effect of LY2828360 and WIN55212-2 on IP1 accumulation via mouse CB₂ receptor.

FIG. 13B is a graph that shows the effect of LY2828360 and WIN55212-2 on IP1 accumulation via human CB₂ receptor.

FIG. 14A is a graph that shows the effect of AM1710 on cAMP levels in HEK cells stably expressing mouse CB₂ receptors.

FIG. 14B is a graph that shows the effect of AM1710 on cAMP levels in HEK cells stably expressing mouse CB₂ receptors over time.

FIG. 14C is a graph that shows the effect of AM1710 on cAMP levels in HEK cells stably expressing human CB₂ receptors after pertussis toxin (PTX) treatment.

FIG. 14D is a graph that shows the effect of AM1710 on cAMP levels in HEK cells stably expressing human CB₂ receptors after pertussis toxin (PTX) treatment.

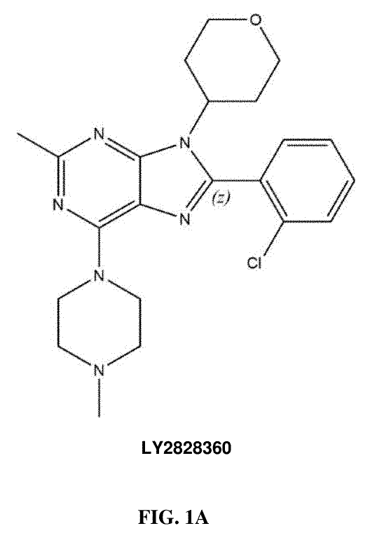

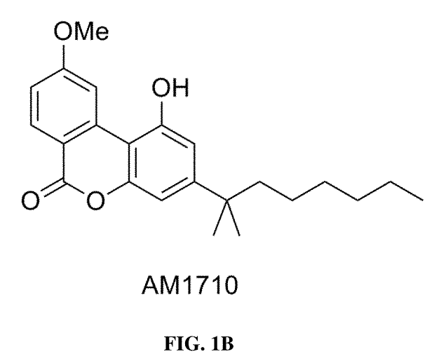

### DETAILED DESCRIPTION

### Definitions

As used herein, the articles, “a”, “an”, and “the” are used herein to refer to one or to more than one (i.e., to at least one) of the grammatical object of the article unless the context clearly and unambiguously dictates otherwise. By way of example, “an element” means one element or more than one element.

As used herein, the term “adjuvant” refers to a combination of therapeutically beneficial agents or active ingredient, including, but not limited to an opioid or a CB₂ receptor agonist, such as AM1710 and LY2828360.

As used herein, an “analog” of a chemical compound is a compound that, by way of example, resembles another in structure but is not necessarily an isomer (e.g., 5-fluorouracil is an analog of thymine).

The term “biocompatible,” as used herein, refers to a material, an agent, a compound, and/or a composition that does not elicit a substantial detrimental response when administered to the subject or host.

The term “biological sample” or “sample,” as used herein, refers to samples obtained from a subject, including, but not limited to, sputum, mucus, phlegm, tissues, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. One of skill in the art will understand the type of sample needed.

As used herein, the term “carrier molecule” refers to any molecule that is chemically conjugated to a molecule of interest.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “compound,” as used herein, refers to any type of substance or agent that comprises, consists essentially of, or consists of an active ingredient, such as LY2828360 or AM1710. A “compound” of the present disclosure is commonly considered a pharmaceutical, a drug, or a candidate for use as a drug, as well as combinations and mixtures of the above, such as LY2828360, AM1710, or combinations thereof.

As used herein, a “derivative” refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, in one embodiment, the term “diagnosis” refers to medical detection of a disease, a disorder, a condition, or a discomfort by a licensed physician. In any method of diagnosis exists false positives and false negatives. Any one method of diagnosis does not provide 100% accuracy.

A “dependence,” as used herein, refers to an uncontrollable physical, mental, emotional overreliance or addiction to a substance or an agent. Illustrative embodiments of the dependence of the present disclosure includes physical dependence. In some embodiments, a subject is physically dependent on a drug or pharmaceutical composition, such as an opioid. Physical and/or clinical dependence is often indicated if and when the subject experiences visible withdrawal signs and/or symptoms due to the reduction or lowering of the concentration of drug in the body of the subject. Common signs or symptoms associated with physical dependence and/or opioid withdrawal include, but are not limited to tremors, chills, goose bumps, day and/or night sweats, nausea, vomiting, diarrhea, sensitivity to light, headaches, cramps, irritation, agitation, muscle aches, runny now, insomnia, dilated pupils, red eyes, withdrawal jumps, etc.

Often, “dependence,” whether physical, mental, or emotional, may be medically diagnosed and/or treated by a licensed physician. An illustrative dependence of the present disclosure comprises an opioid dependence, addiction. Common prescription opioids that a subject may become dependent or addicted to include, but are not limited to morphine, codeine, oxycodone, oxycontin, hydrocodone, methadone, meperidine, buprenorphine, hydromorphone, tapentadol, tramadol, and fentanyl. Notably, heroin is an illegal opioid and common street drug to which many subjects become addicted in the U.S. annually.

“Discomfort,” as described herein, refers to pain. It is commonly known that there are three different types of pain, which are differentiated depending on where they are felt in the body of a subject. The term “pain” of the present disclosure comprises 1) somatic pain, 2) visceral pain, 3) neuropathic pain, or combinations thereof. All three types of pain may be felt by a subject at the same or different times, and all three types of pain may be acute (i.e., short lasting) or chronic (i.e., long-lasting). While acute pain is considered short lasting or intermittent (i.e., lasting less than 90 days consistently or regularly), chronic pain generally lasts more than 90 days or 3 months.

Somatic pain is cause by activation of pain receptors in deep tissue or at the surface. Visceral pain refers to pain on the internal areas of the body that are enclosed with a cavity (e.g., pelvis, chest, abdomen, etc. Most cancer patients experience somatic pain and visceral pain.

The phrase “neuropathic pain,” as described in the present disclosure is typically caused by injury to the central nervous system (CNS). Often, neuropathic pain is a symptom of cancer resulting from tumors pressing or compressing nerves or the spinal cord. Neuropathic pain also occurs when cancer cells actually infiltrate nerves or the spinal cord.

In some subjects and or patients, particularly cancer patients, neuropathic pain may be a result or a side effect of chemotherapy and/or radiation treatment. About 15-20% of cancer patients report neuropathic pain. Accordingly, in one embodiment, neuropathic pain of the present disclosure does not comprise somatic pain or visceral pain. In another embodiment, neuropathic pain of the present disclosure does not comprise somatic pain and visceral pain. In a further embodiment, neuropathic pain of the present disclosure is associated with cancer or cancer therapy, such a chemotherapy and/or radiation. An illustrative example of neuropathic pain of the present disclosure comprises, consisting essentially of, or consisting of neuropathic pain associated with opioid tolerance or neuropathic pain without opioid tolerance.

A “disease” is a state of health of a human or an animal wherein the human or animal cannot maintain homeostasis, and wherein if the disease is not ameliorated then the health of the human or the animal continues to deteriorate, possibly to a point of death.

In contrast, a “disorder” in a human or an animal is a state of health in which the human or the animal is able to maintain homeostasis, but in which the animal's state of health is less favorable than it would be in the absence of the disorder. Left untreated, a disorder does not necessarily cause a further decrease in the state of health of the human or the animal.

As used herein, an “effective amount” or “therapeutically effective amount” means an amount of one or more compounds and/or compositions (e.g., LY2828360 or AM1710) that is sufficient to produce a selected effect, such as alleviating signs and/or symptoms of a disease, a disorder, a dependence or discomfort, such as pain. In the context of administering more than one compound and/or composition in the form of a combination, such as multiple compounds and/or compositions, the amount of each compound and/or composition, when administered in combination with another compound(s) and/or compositions(s), may be different from when that compound is administered alone.

A “ligand” is a compound that specifically binds to a target receptor.

A “receptor” is a compound that specifically binds to a ligand.

A ligand or a receptor (e.g., an antibody or analyte) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample.

As used herein, “parenteral administration” of a pharmaceutical composition includes any route of administration characterized by physical breaching of a tissue of a subject as defined herein, and administration of the pharmaceutical composition to the subject through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “pharmaceutical composition” shall mean a composition comprising at least one active ingredient, whereby the composition is amenable to investigation for a specified, efficacious outcome in a subject, such as a mammal, for example, without limitation, a human or an animal. Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or an analog or derivative thereof that may be combined and which, following the combination, may be used to administer the appropriate or “effective” amount of compound and/or active ingredient to a subject.

As used herein, the phrase “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means that a compound, composition, and/or active ingredient is physiologically tolerable for either human or veterinary/animal application or use.

As used herein, “pharmaceutical compositions” include formulations for human and veterinary use.

A “plurality” means at least two, and may comprise only a few more than two (e.g., 3-5, 3-10, 3-20, 3-100, or more) or many more than two, such as hundreds, thousands, or millions, and a number or amount that is too innumerable to specifically quantify.

A “receptor,” as used herein, is a compound that directly binds to a ligand. Many receptors are cell surface proteins that recognize signals from the exterior of the cell and transduce the signal to the interior of the cell to cause downstream effects and/or functional changes within the cell. Depending on the cell type, different cells may express different and/or different types of cell surface receptors. The term “regulate” refers to either stimulating or inhibiting a function or activity of interest.

A “sample,” as used herein, refers preferably to a biological sample from a subject for which an assay or other use is needed, including, but not limited to, normal tissue samples, diseased tissue samples, sputum, mucus, phlegm, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. A sample can also be any other source of material obtained from a subject who contains cells, tissues, or fluid of interest. A sample can also be obtained from cell or tissue culture.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it may be a known standard agent or compound which is administered and used for comparing results when administering a test compound, or it may be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject” or a “subject in need thereof” is a patient, animal, mammal, or human, who will benefit from the methods and/or compositions of this invention. For example, the subject may be a mammal suffering from pain. The mammal may be an animal, such as a rodent, including, but not limited to a mouse or a rat.

The mammal may also be a human. The human subject or human patient may be female or male, such as a female subject or a male subject or a female patient or a male patient. The human patient may also be a “pre-symptomatic patient,” meaning that the subject or patient has not yet experienced symptoms acknowledged to be associated with a disease, a disorder, or discomfort, such as pain.

The term “symptom,” as used herein, refers to any morbid phenomenon or departure from the normal in structure, function, observation, manifestation (e.g., clinical manifestation), or a sensation experienced by the subject or patient of the present disclosure that may or may not be visible to an observer, including, but not limited to a licensed physician. For example, a headache is a symptom since it is clearly evident or visible to the patient, a doctor, a nurse, and/or other observers.

One symptom, more than one symptom, or a plurality of symptoms may be indicative of a disease, a disorder, or discomfort, such as pain. Symptoms may also be indicative of tolerance or withdrawal from a compound, composition, or an active ingredient, such as an opioid that includes, but is not limited to morphine, codeine, oxycodone, oxycontin, hydrocodone, methadone, meperidine, buprenorphine, hydromorphone, tapentadol, tramadol, fentanyl, and heroin.

In contrast, a “sign” is objective, visible, and/or tangible evidence of a disease, a disorder, or discomfort, such as pain. For example, a bloody nose is a sign since a bloody nose is evident to the patient, a doctor, a nurse, and/or other observers.

A “therapeutic treatment” is a composition and/or compound comprising, consisting essentially of, or consisting of an active ingredient that is administered to a subject who exhibits signs and/or suffers from symptoms of pathology of a disease, a disorder, a discomfort (e.g., pain), a tolerance or a withdrawal from a compound or active ingredient (e.g., LY2828360, AM1710, opioids, or combinations thereof) for the purpose of diminishing or eliminating those signs and/or symptoms.

The terms “treat” or “treatment” as used herein, mean reducing the frequency with which signs and/or symptoms are experienced by a patient or subject. Likewise, the terms “treat” or “treatment” as used herein also refer to the act of administering an agent, a compound, and/or a composition, preferably the composition of the present disclosure, to reduce the frequency with which symptoms and signs are experienced by a subject.

As used herein, the term “control” refers to a sample used in an analytical procedure for comparison purposes, typically to an unknown sample. A control can be “positive” or “negative”. For example, where the purpose of an analytical procedure is to detect a differentially expressed analyte, transcript, and/or polypeptide in cells or tissue of a subject, it is generally preferable to include a positive control, such as a sample from a known subject exhibiting the desired characteristics or expression. A negative control, such as a sample from a known subject, such as an animal or human, lacks the desired characteristics or expression.

As used herein, the term “detecting” is used in the broadest sense to include both qualitative and quantitative measurements of a specific molecule, compound, or active ingredient, for example, measurements of a specific compound analyte.

Unless otherwise specifically explained, all technical and scientific terms used herein have the same meaning as commonly understood by those of ordinary skill in the art that this disclosure belongs. Definitions of common terms in molecular biology maybe found in, for example: Lewin, Genes V, Oxford University Press, 1994; Kendrew et al. (eds.), The Encyclopedia of Molecular Biology, Blackwell Science Ltd., 1994; and Meyers (ed.), Molecular Biology and Biotechnology: A Comprehensive Desk Reference, VCH Publishers, Inc., 1995.

Compounds and Compositions of the Present Methods

A pharmaceutical composition of the methods of the present disclosure comprises, consists essentially of, or consists of one, one or more, two, two or more, or a plurality of a cannabinoid CB₂ receptor agonist compounds. Exemplary embodiments of a cannabinoid CB₂ receptor agonist compound or composition of the present methods comprise, consist essentially of, or consist of a LY2828360 compound, a AM1710 compound, or a combination thereof. The LY2828360 and/or AM1710 compound of the present disclosure may encompass diastereomers and enantiomers of the illustrative compounds.

Generally, the LY282360 compound has a molecular weight of about 426.94 g/mol. The cannabinoid CB₂ receptor agonist and/or LY2828360 compound of the present disclosure may comprise, consist essentially of, or consist of 8-(2-chlorophenyl)-2-methyl-6-(4-methylpiperazin-1-yl)-9-(tetrahydro-2H-pyran-4-yl)-9H-purine. One exemplary embodiment of the cannabinoid CB₂ receptor agonist and/or LY2828360 compound of the present disclosure comprises, consists essentially of, or consists of the following chemical structure or formula:

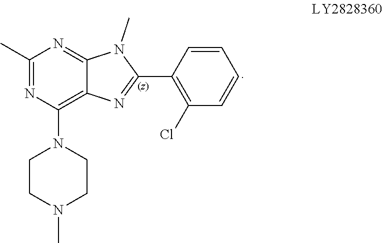 [SMILES: Cc1nc(N2CCN(C)CC2)c2nc(-c3ccccc3Cl)n(C)c2n1]

Another embodiment of the cannabinoid CB₂ receptor agonist compound of the present disclosure comprises, consists essentially of, or consists of an analog, a derivative, a pharmaceutically acceptable salt, a hydrate, a prodrug, or combinations of the LY2828360 compound.

The LY2828360 compound is a G-protein biased compound, meaning that it has the ability to selectively activate G-protein signaling pathways, such as the cAMP and pERK 1/2 pathways. In addition, the LY2828360 compound may activate specific pathways (e.g., cAMP and pERK 1/2 pathways) without activating other unnecessary pathways (e.g., arrestin pathway). Thus, the LY2828360 compound exhibits “biased agonism” and/or “functional selectivity.”

The biased agonism of the LY2828360 compound enables it to selectively activate signaling pathways, such as the cAMP and pERK 1/2 pathways. In particular, the LY2828360 seems to be strongly biased toward G_{i/0}G protein signaling with little effect on arrestin or G_{q} signaling. More specifically, the LY2828360 is capable of selectively activating a signaling pathway that is more therapeutically relevant than another pathway that the compositions does not activate. Thus, the LY2828360 of the present disclosure is a G-protein biased agonist.

In addition, LY2828360 may act in a “slow” manner, or as a “slow-acting” signaling compound. In this regard, the phrases “slow” and “slow-acting” refer to the time in which the LY2828360 compound is able to slow or inhibit adenyl cyclase. Further, the phrases “slow” and “slow-acting” refer to the ability and/or capability of the LY2828360 compound to activate related G-protein signaling pathways (e.g., cAMP and pERK 1/2 pathways) within a time period of about 15 minutes to about 60 minutes.

For example, the LY2828360 compound may activate related G-protein signaling pathways in a time frame that ranges from about 20 minutes to about 55 minutes, about 25 minutes to about 50 minutes, about 30 minutes to about 45 minutes, about 30 minutes to about 40 minutes, about 30 minutes to about 35 minutes, and at or about 30 minutes. In an exemplary embodiment, the LY2828360 compound may activate related G-protein signaling pathways in a “slow” timeframe of about 30 minutes.

Typically, the AM1710 compound has a molecular weight of about 369 g/mol. The cannabinoid CB₂ receptor agonist and/or the AM1710 compound of the present disclosure may comprise, consist essentially of, or consist of 3-(1,1-dimethyl-heptyl)-1-hydroxy-9-methoxy-benzo(c) chromen-6-one. One exemplary embodiment of the cannabinoid CB₂ receptor agonist and/or AM1710 compound of the present disclosure comprises, consists essentially of, or consists of the following chemical structure or formula:

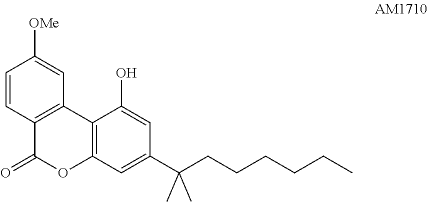 [SMILES: CCCCCCC(C)(C)c1cc(O)c2c(c1)oc(=O)c1ccc(OC)cc12]

Another embodiment of the cannabinoid CB₂ receptor agonist compound of the present disclosure comprises, consists essentially of, or consists of an analog, a derivative, a pharmaceutically acceptable salt, a hydrate, a prodrug, or combinations of the AM1710 compound.

AM1710 is a cannabilactone CB₂ receptor agonist. Similar to other standard CB₂ agonists, such as CP55940, the AM1710 compound is a functionally balanced cannabinoid agonist. As such, the AM1710 compound has the ability to activate several G-protein signaling pathways and non-G-protein signaling pathways together, whether simultaneously or consecutively. For example, the AM1710 compound may activate multiple pathways, such as G-protein signaling pathways (e.g., cAMP and pERK 1/2 pathways), along with the arrestin pathway. Thus, the AM1710 compound exhibits “functional balance,” and does not seem to be a G-protein biased agonist. Instead, the AM1710 compound is a functionally balanced agonist.

In addition, AM1710 may act in “fast” manner, or as a “fast-acting” signaling compound. In this regard, the phrases “fast” and “fast-acting” refer to the time in which the AM1710 compound is able to inhibit adenyl cyclase. Further, the phrases “fast” and “fast-acting” refer to the ability and/or capability of the AM1710 compound to activate multiple signaling pathways, including G-protein and non-G-protein signaling pathways (e.g., cAMP, pERK 1/2, and arrestin pathways) within a time period of about 0.5 minutes to about 12 minutes.

For example, the AM1710 compound may activate dual (2) and/or multiple signaling pathways in a time frame that ranges from about 1 minutes to about 11 minutes, about 2 minutes to about 10 minutes, about 3 minutes to about 9 minutes, about 4 minutes to about 8 minutes, about 5 minutes to about 7 minutes, about 4.5 minutes to about 6 minutes, about 5 minutes to about 5.5 minutes, and at or about 5 minutes. In an exemplary embodiment, the AM1710 compound may activate multiple signaling pathways in a “fast” timeframe of about 5 minutes. The LY2828360 and AM1710 compounds, along with any analogs, derivatives, pharmaceutically acceptable salts, hydrates, prodrugs, and/or combinations thereof, may be prepared synthetically. In some embodiments, the analogs, derivatives, pharmaceutically acceptable salts, hydrates, prodrugs, and/or combinations of the LY2828360 or AM1710 compounds or compositions have increased stability, decreased oxidation, and/or increased half-life than the compound itself. In other embodiments, the analogs, derivatives, pharmaceutically acceptable salts, hydrates, prodrugs, and/or combinations thereof of the LY2828360 or AM1710 compounds or compositions target the LY2828360 and AM1710 compounds or composition to a specific cell and/or tissue.

In some embodiments, any of the compositions or compounds described herein can be modified with a prodrug to prolong half-life. Prodrugs may also be helpful to protect the compound or composition of the present disclosure against oxidation, degradation, to target the compound to a tissue. Alternatively, prodrugs may help allow the compound or compositions of the present disclosure to pass the blood brain barrier.

Compounds and compositions of the present disclosure may comprise, consist essentially of, or consist of any amount of active ingredient (e.g., LY2828360 or AM1710) that is effective to treat a disease, a disorder, or a chronic discomfort, such as pain or neuropathic pain. More specifically, effective concentrations of the compounds and/or compositions of the present disclosure may comprise any amount of LY2828360 or AM1710 compounds that is effective to treat pain, such as neuropathic pain, without tolerance. For example, in some embodiments, an effective composition of the present methods comprises, consists essentially of, or consists of at least about 0.1 mg/kg i.p. of the LY2828360 or

AM1710 compounds, or combinations thereof.

In other embodiments, an effective composition of the present methods comprises, consists essentially of, or consists of at least about 0.05 mg/kg i.p. of the LY2828360 or AM1710 compounds. In particular, an effective composition of the present disclosure may comprise, consist essentially of, or consist of a range of about 0.01 mg/kg i.p. to about 15 mg/kg i.p. of the LY2828360 or AM1710 compounds, and all percentage values within that range.

In some embodiments, the effective amount or concentration of the composition of the present disclosure comprises, consists essentially of, or consists of about 0.01 mg/kg i.p. to about 10 mg/kg of the LY2828360 compound, and all percent range values in between. In further embodiments, the effective amount or concentration of the compositions of the present methods comprises, consists essentially of, or consists of about 0.05 mg/kg i.p. to about 9 mg/kg, 0.075 mg/kg i.p. to about 7 mg/kg, 0.05 mg/kg i.p. to about 5 mg/kg, 0.025 mg/kg i.p. to about 4 mg/kg, about 0.05 mg/kg i.p. to about 3 mg/kg, about 0.1 mg/kg i.p. to about 3 mg/kg, about 0.075 mg/kg i.p. to about 2 mg/kg, about 0.090 mg/kg i.p. to about 1 mg/kg, about 0.085 mg/kg i.p. to about 0.75 mg/kg, about 0.070 mg/kg i.p. to about 0.5 mg/kg, about 0.06 mg/kg i.p. to about 0.4 mg/kg, about 0.1 mg/kg i.p. to about 0.3 mg/kg, about 0.065 mg/kg i.p. to about 0.2 mg/kg, about 0.06 mg/kg i.p. to about 0.2 mg/kg, and about 0.07 mg/kg i.p. to about 0.1 mg/kg of the LY2828360 compound.

In some embodiments, the effective amount or concentration of the composition of the present disclosure comprises, consists essentially of, or consists of about 0.01 mg/kg i.p. to about 15 mg/kg of the AM1710 compound, and all percent range values in between. In further embodiments, the effective amount or concentration of the compositions of the present methods comprises, consists essentially of, or consists of about 0.05 mg/kg i.p. to about 14 mg/kg, 0.075 mg/kg i.p. to about 13 mg/kg, 0.05 mg/kg i.p. to about 12 mg/kg, 0.025 mg/kg i.p. to about 11 mg/kg, about 0.1 mg/kg i.p. to about 10 mg/kg, about 0.5 mg/kg i.p. to about 9 mg/kg, about 0.75 mg/kg i.p. to about 8 mg/kg, about 1 mg/kg i.p. to about 7 mg/kg, about 1.5 mg/kg i.p. to about 6 mg/kg, about 2 mg/kg i.p. to about 5 mg/kg, about 2.5 mg/kg i.p. to about 4 mg/kg, about 2 mg/kg i.p. to about 10 mg/kg, about 2.25 mg/kg i.p. to about 13 mg/kg, about 2.3 mg/kg i.p. to about 12 mg/kg, and about 2.5 mg/kg i.p. to about 10 mg/kg of the AM1710 compound.

In further embodiments, the effective amount or concentration of the composition of the present disclosure comprises, consists essentially of, or consists of at, about, or at least 0.1 mg/kg, 0.3 mg/kg, or 3 mg/kg i.p. of the LY2828360 or AM1710 compounds. In other embodiments, the effective amount or concentration of the composition of the present disclosure comprises, consists essentially of, or consists of no greater than about 3 mg/kg i.p. of the LY2828360 compound or about 10 mg/kg i.p. of the AM1710 compound, or combinations thereof.

According to another aspect of the present invention, a pharmaceutical composition comprises, consist essentially of, or consists of a therapeutically-effective amount (also called “the effective amount”) of one or more compounds of the present invention (e.g., LY2828360 and AM1710) or a pharmaceutically acceptable salt, ester, analyte, derivative, or prodrug thereof, together with a pharmaceutically acceptable diluent or a pharmaceutically acceptable carrier (i.e., “a carrier”). Carriers of the present disclosure are materials or compositions involved in carrying or transporting an active ingredient or compound (e.g., LY2828360 and AM1710), including any analogs, derivatives, pharmaceutically acceptable salts, hydrates, prodrugs, and/or combinations thereof from one location to another location. Carriers may be combined with an active cannabinoid CB₂ receptor agonist compound (e.g., LY2828360 or AM1710) of the present disclosure to form a compound treatment. Treatment carriers of the present disclosure may comprise liquids, gases, oils, solutions, solvents, solids, diluents, encapsulating materials, or chemicals.

For example, a liquid carrier of the present disclosure may comprise water, buffer, saline solution, a solvent, etc. In some embodiments, pharmaceutically acceptable carriers may include water, physiological saline, and/or aqueous buffered solutions that may or may not comprise surfactants or stabilizing amino acids, such as histidine or glycine. In other embodiments, a pharmaceutically acceptable carrier may comprise liquid carriers, such as physiological saline, ethanol, dimethyl sulfoxide (DMSO), castor oil ethoxylate, or combinations thereof. For example, the carrier may comprise, consist essentially of, or consist of a combination of DMSO, castor oil ethoyxlate (e.g., ALKAMULS EL-620, Solvay), ethanol, and saline at a ratio of 2:1:1:18, respectively. In one embodiment of the present application, the pharmaceutically acceptable carrier is pharmaceutically inert.

In some embodiments of the present disclosure, compositions and/or formulations comprising the active ingredient (e.g., LY2828360 or AM1710) may be administered alone or in combination with other forms of active ingredients, drugs, pharmaceuticals, and/or small molecules. In some embodiments, compositions and methods of the present disclosure may comprise, consist essentially of, or consist of active ingredients (e.g., LY2828360 or AM1710) in combination with one or more opioids. Opioids of the present methods may comprise, consist essentially of, or consist of morphine, codeine, oxycodone, oxycontin, hydrocodone, methadone, meperidine, buprenorphine, hydromorphone, tapentadol, tramadol, fentanyl, heroin, and/or combinations thereof. An exemplary opioid of the present compositions and methods is morphine. In some embodiments, the compositions and methods of the present disclosure may comprise LY2828360, AM1710, morphine, and/or combinations thereof.

Alternatively, the active ingredient or compounds of the present disclosure (e.g., LY2828360 or AM1710) may be comprised in pharmaceutical compositions where it is mixed with excipient(s) or other pharmaceutically acceptable carriers. For example, compositions of the present application can be formulated using pharmaceutically acceptable carriers well known in the art in dosages suitable for oral administration. The carriers may enable the pharmaceutical compositions to be formulated as tablets, pills, capsules, liquids, gels, syrups, slurries, suspensions and the like, or for oral or nasal ingestion by a subject to be treated.

In addition to carriers, other components may be comprised in the composition and/or compound treatment of the present disclosure. Additional components of the present compound treatment and/or compositions may include, but are not limited to adjuvants, surfactants, excipients, dispersants, emulsifiers, etc. In particular embodiment, such additional components may be comprised in the present compound treatment or compositions including dimethylsulfoxide (DMSO), Alkamuls EL-620, ethanol and saline in a ratio of 2:1:1:18

Generally, the cannabinoid CB₂ receptor agonist treatment complex, composition, compound, and/or active ingredient of the claimed methods may be an experimental and/or clinical therapeutic composition. The cannabinoid CB₂ receptor agonist treatment complex of the claimed methods is used for treating and/or relieving one or more symptoms, signs, and/or one or more clinical manifestation of a dependence or a discomfort in a subject. The treatment composition or complex of the claimed methods comprises, consists essentially of, or consists of cannabinoid CB₂ receptor agonist compounds of the present disclosure (e.g., LY2828360 or AM1710).

Methods of the Present Disclosure

The present methods utilize pharmaceutical and biological methodologies to administer constructs, compositions, and/or components in order to effect positive change for subjects that suffer from a disease, a disorder, a dependence (e.g., to opioids, such as morphine), a discomfort (i.e., pain), a tolerance (e.g., to opioids, such as morphine), or a withdrawal from opioids. Therefore, the claimed methods have direct application to treatment of opioid dependence, tolerance, withdrawal, and discomfort, such as neuropathic pain.

Opioids of the present disclosure include, but are not limited to narcotics that are abused by people. Illustrative opioids of the present methods may comprise, consist essentially of, or consist of, morphine, codeine, oxycodone, oxycontin, hydrocodone, methadone, meperidine, buprenorphine, hydromorphone, tapentadol, tramadol, fentanyl, and heroin. An exemplary opioid is of the present methods is morphine.

Thus, one embodiment of the methods of the present disclosure relates to treating pain (e.g., neuropathic pain) in a subject. In another embodiment, the method of treating pain comprises suppressing or attenuating neuropathic pain in a subject. In a further embodiment, the method of treating pain comprises suppressing or attenuating neuropathic pain in a subject without opioid tolerance.

The method of treating pain (e.g., neuropathic pain) without opioid tolerance of the present disclosure comprises, consists essentially of, or consists of administering a pharmaceutical composition comprising one or more or a plurality of an active cannabinoid CB₂ receptor agonist compound (e.g., LY2828360 or AM1710) to the subject. The method further comprises improving one or more clinical manifestations of neuropathic pain in the subject, such as suppressing, blocking, delaying, and/or preventing opioid tolerance.

For example, in one embodiment, the LY2828360 compound may be used as a slow signaling cannabinoid CB₂ G-protein biased receptor agonist to suppress neuropathic pain, while preventing and/or totally suppressing opioid tolerance with no loss of efficacy over time, and preventing or suppressing opioid withdrawal. In another embodiment, the AM1710 compound may be used as a fast signaling balanced cannabinoid CB₂ receptor agonist to suppress neuropathic pain, suppress opioid withdrawal, or delay opioid tolerance. However, in this embodiment, the AM1710 compound does not totally prevent or suppress opioid tolerance, as does the LY2828360 compound. Instead, the AM1710 compound works to delay opioid tolerance while treating neuropathic pain.

Further, the methods of the present disclosure comprise, consist essentially of, or consist of inhibiting cyclic AMP (cAMP) accumulation in cells of the subject. Inhibition of the cAMP pathway prevents, delays, suppresses, or reverses one or more clinical manifestations of neuropathic pain, such as chemotherapy-induced neuropathic pain in the subject. In a further embodiment, the methods of the present disclosure is related to activating phosphorylated ERK1/2 (pERK 1/2) in a subject. More specifically, the LY2828360 compound may actively select a pathway that affects neuropathic pain, such as that experienced by patients subject to chemotherapy treatments for cancer.

Alternatively, while the cannabinoid CB₂ receptor agonist compounds (e.g., LY2828360 and AM1710) of the present disclosure may be effective to treat neuropathic pain, they may also be ineffective at relieving other types of pain (e.g., normal, inflammatory pain, and/or pain due to an injury). For example, in some embodiments, the LY2828360 compound was ineffective in relieving pain associated with osteoarthritis, which is not a neuropathic pain, but is instead an inflammatory pain. Accordingly, the cannabinoid CB₂ receptor agonist compounds of the present disclosure (e.g., LY2828360 and AM1710) may be selectively biased toward treatment of neuropathic pain versus other types of pain, such as normal pain, inflammatory pain, and/or pain due to an injury.

Therefore, the present methods treat one or more clinical manifestations of neuropathic pain, such as chemotherapy-induced neuropathic pain, opioid tolerance, and/or opioid dependence in the subject. Moreover, the present methods suppress neuropathic pain without producing a tolerance, such as the tolerance observed in users of morphine (i.e., morphine tolerance) often requiring such users to consistently required increased doses of morphine to obtain the same effect on pain relief. In particular, the active cannabinoid CB₂ receptor agonist compound (e.g., LY2828360 and AM1710) of the present disclosure, when administered to a subject alone or co-administered with another compound and/or composition (e.g., morphine), strongly attenuates, reduces, delays, and/or prevents development of tolerance to opioids, such as morphine. The present methods also decrease, suppress, and/or prevent naloxone-precipitated withdrawal signs and/or symptoms in subjects treated with the cannabinoid CB₂ receptor agonist compound (e.g., LY2828360 or AM1710) of the present disclosure.

Another embodiment of a method of the present disclosure is related to a method of monitoring efficacy of cannabinoid CB₂ receptor agonist compound (e.g., LY2828360 or AM1710) treatment in a subject. The method comprises measuring one or more clinical manifestations of pain and/or morphine tolerance, withdrawal, and/or dependence, particularly with references to measures of neuropathic pain, mechanical and cold allodynia, and/or tissues of a subject prior to treatment administration. The method further comprises administering a treatment composition comprising one or more or a plurality of cannabinoid CB₂ receptor agonist compounds (e.g., LY2828360 or AM1710) to the subject. At least 5 minutes, 20 minutes, 40 minutes, 60 minutes (i.e., one hour), 24 hours, 48 hours, and more after treatment administration, the method optionally comprises remeasuring the one or more clinical manifestations of pain in the subject. Additionally, the method further comprises assessing the one or more clinical manifestations of pain (e.g., neuropathic pain) by determining the difference between the cells and/or tissues of the subject prior to treatment administration compared to the cells and/or tissues of the subject after treatment administration.

Another method encompassed by the present disclosure is one or more methods of diagnosing, prognosing, and/or monitoring the progression of a neuropathic pain in a subject or a patient. The method comprises assessing, measuring, and/or quantitating the sign and/or symptoms and or clinical manifestations, including secondary effects, of the disease in the subject, if applicable. Notably, the subject or patient may be pre-symptomatic, such that there are no symptoms and/or clinical manifestations of disease to initially assess. One or more clinical manifestations, signs, or “symptoms” of the disorder or discomfort of pain treated by the present methods as experienced by the subject comprise, consist essentially of, or consist of hypersensitivity to mechanical and cold stimulation, referred to herein as mechanical and cold allodynia, respectively, and/or combinations thereof.

Additional diagnostic and/or mechanistic methods are also described in the present disclosure. For example, one embodiment off the present methods is directed to a method to detect and correct present and/or potential defects in the treatment of pain, such as neuropathic pain, in a subject. The method comprises utilizing, testing, experimenting, dosing, and/or investigating a mouse model of pain or morphine dependence to identify, detect, and/or correct any problems in treating humans for pain, opioid tolerance, dependence and/or withdrawal. This method of utilizing mouse models for pain would also enable identification of molecular, genetic, biomarkers, and/or selectable markers to assess the efficacy of cannabinoid CB₂ receptor agonist compounds (e.g., LY2828360) therapy in humans.

Finally, the methods of the present disclosure comprise a method of administering the cannabinoid CB₂ receptor agonist compounds and compositions of the present disclosure. More specifically, methods of administering the present cannabinoid CB₂ receptor agonist compounds and compositions (e.g., LY2828360 and AM1710) to a subject have shown clinical effect and efficacy in treating neuropathic pain, reducing opioid withdrawal signs and symptoms (e.g. a plurality of withdrawal jumps), and/or reducing or preventing development of opioid tolerance. In particular, the method of reducing or preventing development of opioid tolerance comprises suppressing, preventing, delaying, or mitigating development of presentation of one or more clinical manifestations of opioid tolerance. The present methods have been demonstrated in living subjects, such as mice and/or humans.

Thus, the present cannabinoid CB₂ receptor agonist compounds and compositions (e.g., LY2828360 and AM1710) may be administered in vitro, in vivo, and/or ex vivo. For example, the compounds and compositions of the present disclosure (e.g., LY2828360 and AM1710) may be administered in vitro to cells including, but not limited to human embryonic kidney (HEK) cells and CHO cells. When administered in vivo, the cannabinoid CB₂ receptor agonist compounds of the present disclosure (e.g., LY2828360 and AM1710) may be administered to one or more subjects in order to treat diseases, disorders, dependence, or discomfort (e.g., neuropathic pain).

Subjects of the present composition comprise, consist essentially of, or consist of human subjects and/or veterinary subjects. Human subjects may comprise, consist essentially of, or consist of humans that are or are not afflicted with a disease, a disorder, or discomfort, such as pain. In an exemplary embodiment, a human subject is a human patient being a person that may be suffering from a disease, a disorder, or discomfort, such as pain. More specifically, a human subject of the present disclosure may comprise, consist essentially of, or consist of a human or a person that is suffering from pain, such as neuropathic pain.

Alternatively, veterinary subjects of the present invention include, but are not limited to any type, kind, species, or breed of a domestic, wild, or laboratory animal. Illustrative embodiments of veterinary subjects may comprise, consist essentially of, or consist of mice, dogs, rabbits, rats, guinea pigs, and any other type of animal. An exemplary embodiment of a veterinary subject is a mouse or a plurality of mice. Particular embodiments of a mouse of the present disclosure include, but are not limited to wildtype, Paclitaxel-treated wildtype, CB₂ Knockout (CB₂KO) mice, and other species.

As is well known in the medical arts, dosages of a compound or composition comprising an active ingredient (e.g., LY2828360 or AM1710) for any one subject may depend upon many factors, including the subject's size, body surface area, age, the particular compound to be administered, sex, time and route of administration, general health, and interaction with other drugs being concurrently administered. Depending on the target sought to be altered by treatment, pharmaceutical compositions of the present disclosure may be formulated and administered systemically or locally.

Techniques known in the art for formulation and administration of therapeutic compounds are sufficient to administer the compounds and compositions of the present invention. The compositions of the present disclosure may be formulated for any route of administration, in particular for oral, rectal, transdermal, subcutaneous, intravenous, intramuscular or intranasal administration. Suitable routes of administration may, for example, include oral or transmucosal administration; as well as parenteral delivery, including intramuscular, subcutaneous, intramedullary, intrathecal, intraventricular, intravenous, intraperitoneal, or intranasal administration.

For injection, a composition of the present application (e.g., LY2828360 or AM1710 compounds) may be formulated in aqueous solutions, such as in physiologically compatible buffers such as Hanks' solution, Ringer's solution, or physiologically buffered saline. For tissue or cellular administration in a subject, penetrants of the present compounds and/or composition appropriate to the particular barrier to be permeated are used in the formulation. Such penetrants are generally known in the art.

Importantly, the treatment complex of the present disclosure may be administered in an experimental, research, medicinal, or clinical environment to a human subject as a therapeutic composition. The therapeutic composition of the present disclosure may also include an adjuvant or a pharmaceutically acceptable carrier. In one aspect, cannabinoid CB₂ receptor agonist compounds and compositions (e.g., LY2828360 or AM1710) are included in the therapeutic composition. Various aspects and embodiments of methods of administering the treatment construct of the present disclosure are described in further detail below.

Another embodiment of the present disclosure is directed to methods of preparation and use (i.e., administration) of a pharmaceutical composition of the present disclosure. The therapeutic or compound of the present disclosure comprises, consists essentially of, or consists of a cannabinoid CB₂ receptor agonist compounds and compositions (e.g., LY2828360 or AM1710) useful for the treatment of diseases, disorders, dependence, and/or discomfort, such as neuropathic pain, as disclosed herein. In the present pharmaceutical or therapeutic construct, an LY2828360 or AM1710 may be the compound or active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these.

Pharmaceutically acceptable carriers include physiologically tolerable or acceptable diluents, excipients, solvents or adjuvants. The compositions are preferably sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical or therapeutic compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions may be prepared in conventional forms, either as liquid solutions or suspensions, solid forms suitable for solution or suspension in liquid prior to injection, or as emulsions. Pharmaceutical compositions according to the present invention may be prepared in a manner fully within the skill of the art.

The therapeutic or pharmaceutical composition of the present disclosure may include pharmaceutically acceptable salts thereof, or pharmaceutical compositions comprising these compounds may be administered so that the compounds may have a physiological effect. For example, in one embodiment of the present disclosure, a protein or peptide of the invention, or a combination thereof, may be administered to a subject by a route selected from, including, but not limited to, intravenously, intrathecally, locally, intramuscularly, topically, orally, intra-arterially, etc. Administration may also occur enterally or parenterally; for example orally, rectally, intracisternally, intravaginally, intraperitoneally, locally (e.g., with powders, ointments or drops), or as a buccal or nasal spray or aerosol. Parenteral administration is preferred. Particularly preferred parenteral administration methods include intravascular administration (e.g. intravenous bolus injection, intravenous infusion, intra-arterial bolus injection, intra-arterial infusion and catheter instillation into the vasculature), peri- and intra-target tissue injection (e.g. peri-tumoral and intra-tumoral injection), subcutaneous injection or deposition including subcutaneous infusion (such as by osmotic pumps), intramuscular injection, and direct application to the target area, for example by a catheter or other placement device. Controlled- or sustained-release formulations of a pharmaceutical composition of the invention may be made using conventional technology

The pharmaceutical compositions useful for practicing the invention (i.e., LY2828360 or AM1710) may be administered to deliver a dose of between about 0.03 mg/kg to about 10 mg/kg, and more specifically, from about 0.1 mg/kg to about 3 mg/kg. In some embodiments, the effective dose of the LY2828360 or AM1710 compounds administered to the subject is no greater than 3 mg/kg.

Where the administration of the active ingredient (i.e., LY2828360 or AM1710) is by injection or direct application, the injection or direct application may be in a single dose or in multiple doses. A pharmaceutical composition of the invention may also be prepared, packaged, and/or sold in bulk, such as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

However, where the administration of the compound is by infusion, the infusion may be a single sustained dose over a prolonged period of time or multiple infusions. Notably, an exemplary embodiment of the methods of the present disclosure comprise as few as only a single administration of the treatment or therapeutic composition to a subject or patient without the need for multiple administrations or infusions for the subject to achieve and maintain efficacy of the treatment.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In addition to ex vivo administration of the present compositions, the present disclosure also describes in vivo methods of treating a subject. The methods described herein comprise, consist of, and consist essentially of administering a pharmaceutical or therapeutic composition of the present disclosure comprising at least one compound of the present invention to a subject. In particular, the methods of the present disclosure are directed to administering a cannabinoid CB₂ receptor agonist compounds and compositions (e.g., LY2828360) described herein to a subject for treatment of a disease, disorder, dependence, or discomfort. More specifically, the compositions and methods of the present disclosure are directed to a method of treating neuropathic pain or opioid tolerance (e.g., morphine) by administering the compounds and compositions of the present disclosure to a subject.

Compounds (e.g., LY2828360) identified by the methods of the invention may be administered with known compounds (e.g., morphine) or in combination with other medications as well (e.g., paclitaxel, naloxone, morphine, and CP55940). In accordance with one embodiment, a method of treating pain, such as neuropathic pain, in a subject or a patient is provided wherein the method comprises administering LY2828360, as disclosed herein to the patient.

Typically, dosages of the compound or active ingredient of the invention which may be administered to an animal, preferably a human, in an amount that ranges from 0.03 mg/kg to about 10 mg/kg, and more specifically, from about 0.1 mg/kg to about 3 mg/kg (up to 0.09 mg daily in a 30 g mouse). While the precise dosage administered will vary depending upon any number of factors, including but not limited to, the type of animal and type of disease state being treated, the age of the animal and the route of administration. In one aspect, the dosage of the compound will vary from about 0.1 mg to about 3 mg per kilogram of body weight of the animal. In another aspect, the dosage will vary from about 0.03 mg to about 10 mg per kilogram of body weight of the animal.

One major benefit of the methods of the present disclosure is single administration of the treatment or therapeutic composition of the present method to the patient or subject to achieve and/or maintain efficacy (i.e., reduction and/or prevention of clinical manifestations of pain). However, if necessary, the compound may be administered to a subject (e.g., an animal or human) as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered.

Suitable preparations of the pharmaceutical compositions described herein include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients may also be mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

In addition to the active ingredient, a pharmaceutical composition of the invention may further comprise one or more additional pharmaceutically active or inactive components or agents. Additional ingredients may include, but are not limited to, one or more of the following: excipients; surface active agents; dispersing agents; inert diluents; granulating and disintegrating agents; binding agents; lubricating agents; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other additional ingredients that may be included in the pharmaceutical compositions of the invention are known in the art and described, for example in Genaro, ed., 1985, Remington's Pharmaceutical Sciences, Mack Publishing Co., Easton, Pa.

In other embodiments, therapeutic agents and pharmaceutical compositions of the present disclosure, include, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the compositions described herein. Drugs useful in the invention may, for example, possess a pharmaceutical property selected from the group consisting of antimitotic, antikinase, alkylating, antimetabolite, antibiotic, alkaloid, anti-angiogenic, pro-apoptotic agents, and combinations thereof. Techniques for detecting and measuring these agents are provided in the art or described herein.

Other embodiments of the invention will be apparent to those skilled in the art based on the disclosure and embodiments of the invention described herein. It is intended that the specification and examples be considered as exemplary only, with a true scope and spirit of the invention being indicated by the following claims. While some representative experiments have been performed in test animals, similar results are expected in humans. The exact parameters to be used for injections in humans may be easily determined by a person skilled in the art. Other techniques known in the art may be used in the practice of the present invention.

The invention is now described with reference to the following Examples. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure.

Therefore, the examples should be construed to encompass any and all variations which become evident as a result of the teaching provided herein.

### EXAMPLES

When administered to a subject, such as a human or an animal subject, LY2828360 is a potent CB₂ receptor agonist having similar affinity for human and rat CB₂ receptors. In human CB₂ functional assays, approximately 87% maximal stimulation of CB₂ was observed at 20 nM concentrations of LY2828360, whereas only 15% maximal stimulation of CB₁ was observed at 100 μM concentrations of LY2828360. LY2828360 also shows good central nervous system (CNS) penetration and potent oral activity in preclinical models of joint pain induced by intra-articular monoiodoacetic acid.

In monoiodoacetic acid models, LY2828360 (0.3 mg/kg p.o.) produced a dose-related reversal of pain using incapacitance testing, demonstrating equivalent efficacy to the nonsteroidal anti-inflammatory drug diclofenac. No specific risks or discomforts associated with LY2828360 were observed in human subjects with osteoarthritic pain who have taken LY2828360 up to a dose of 80 mg for 4 weeks. Unfortunately, LY2828360 and placebo treatments did not differ in achieving the primary endpoint in patients with osteoarthritic knee pain in this phase 2 clinical trial. Evaluations of LY2828360 antinociceptive efficacy have not appeared in the published literature despite that LY2828360-associated improvements were noted in exploratory pain models.

While the signaling profile of LY2828360 was previously unknown, characterization of the signaling of LY2828360 with stably expressed mouse and human CB₂ receptors was performed. More specifically, cell-based in vitro signaling assays, including arrestin recruitment, CB₂ receptor internalization, inhibition of forskolin-stimulated cAMP (cyclase) accumulation, extracellular signal-regulated kinase (ERK1/2) phosphorylation, and myo-inositol phosphate 1 (IP1) accumulation were assessed herein to better understand the clinical characteristics and efficacy of LY2828360 on pain, particularly neuropathic pain. Accordingly, LY2828360 was evaluated in animal models of neuropathic pain.

The same Paclitaxel model of peripheral neuropathy as described herein was used to evaluate whether the LY2828360 and AM1710 compounds. More specifically, the CB₂ receptor agonist, AM1710, suppressed neuropathic pain induced by the chemotherapeutic agent, Paclitaxel, through a CB₂-specific mechanism without producing tolerance or physical dependence in the subject. Similarly, the LY2828360 compound was evaluated to determine whether it would suppress chemotherapy-induced neuropathic pain in a CB₂-dependent manner using both CB₂KO and WT mice. Repeated administration of LY2828360 was also investigated to determine if it would produce tolerance to the antinociceptive effects of the CB₂ agonist in paclitaxel-treated mice. Comparisons were made between LY2828360 and the opioid analgesic, morphine, administered under identical conditions.

In addition, LY2828360 and AM1710 compounds were investigated to determine if they would produce antiallodynic efficacy in subjects that were rendered tolerant to morphine. Conversely, LY2828360 and AM1710 compounds were investigated to determine whether development of morphine tolerance would be attenuated in subjects with a history of chronic LY2828360 and AM1710 compound treatments, respectively. Coadministration of a low dose of LY2828360 or AM1710, with a maximally efficacious dose of an opioid (e.g., morphine), was also investigated to determine if it would attenuate morphine tolerance.

In all studies, pharmacologic specificity was established using wildtype (WT) and CB₂KO mice. Finally, to assess physical dependence, mice where injected with vehicle or the opioid antagonist naloxone to evaluate whether the LY2828360 and AM1710 compounds would impact naloxone-precipitated opioid withdrawal in mice previously rendered tolerant to morphine. Additional studies to investigate the effect of the LY2828360 and AM1710 compounds were further conducted in human cells, tissues, and/or living rodent subjects.

Subjects

Adult male CB₂KO mice [B6.129P2-CNR2 (tm1 Dgen/J), bred at Indiana University] and WT mice (bred at Indiana University or purchased from Jackson Laboratory, Bar Harbor, Me.) on a C57BL/6J background, weighing 25-35 g, were used in this study. Animals were single-housed several days before initiating pharmacologic manipulations. All mice were maintained in a temperature-controlled facility (73±2° F., 45% humidity, 12-hour light/dark cycle, lights on at 7 AM); food and water were provided ad libitum.

Drugs and Chemicals.

Paclitaxel (Tecoland Corporation, Irvine, Calif.) was dissolved in a cremophor-based vehicle made of Cremophor EL (Sigma-Aldrich, St. Louis, Mo.), ethanol (Sigma-Aldrich), and 0.9% saline (Aqualite System; Hospira, Inc., Lake Forest, Ill.) at a ratio of 1:1:18.

LY2828360 (8-(2-chlorophenyl)-2-methyl-6-(4-methylpiperazin-1-yl)-9-(tetrahydro-2H-pyran-4-yl)-9H-purine) was obtained from Eli Lilly and company (Indianapolis, Ind.) and synthesized by Eli Lilly (Indianapolis, Ind.) as previously described.

CP55940 [(2)-cis-3-[2-hydroxy-4-(1,1-dimethylheptyl)phenyl]-trans-4-(3-hydroxypropylcyclohexanol] was obtained from the National Institute of Drug Abuse Drug Supply Service (Bethesda, Md.). Pertussis toxin (PTX; cat. no. BML-G100-0050) was purchased from Enzo Lifesciences (Farmingdale, N.Y.).

AM1710 was synthesized in the laboratory of Alexandros Makriyannis (Northwestern University, Boston, Mass.); CP55940 was purchased from Cayman Chemical Company (Ann Arbor, Mich.) or was obtained from the National Institute of Drug Abuse Drug Supply Service (Bethesda, Md.).

Morphine (Sigma-Aldrich), AM1710, CP55940, or LY2828360 were dissolved in a vehicle containing a 2:1:1:18 ratio of dimethylsulfoxide (DMSO) (Sigma-Aldrich), ALKAMULS EL-620 (Rhodia, Cranbuiy, N.J.), ethanol, and saline. Naloxone (Sigma-Aldrich) was dissolved in saline as indicated.

Drugs were administered via intraperitoneal injection to mice in a volume of or 10 ml/kg.

Cell Culture

Human embryonic kidney (HEK) 293 cells stably expressing mouse CB2 receptors (HEK mCB2) or human CB2 receptors (HEK hCB2) were generated, expanded, and maintained in Dulbecco's modified Eagle's medium with 10% fetal bovine serum and penicillin/streptomycin (GIBCO, Carlsbad, Calif.) at 37° C. in 5% CO₂. For ease of immunodetection, an amino-terminal hemagglutinin epitope tag was introduced into the CB1 and CB2 receptors.

Arrestin Recruitment

To determine arrestin recruitment, assays were performed using an enzyme complementation approach. PathHunter Chinese hamster ovary (CHO) K1CNR2 (cat. no. 93-0472C2) cells were purchased from DiscoveRx (Fremont, Calif.). This cell line is engineered wherein an N-terminal deletion mutant of β-galactosidase (β gal) enzyme acceptor is fused with arrestin while a complementary smaller fragment (C-terminal) is fused with C-terminal domain of the mouse CB₂ cannabinoid receptor. Upon receptor activation, recruitment of arrestin leads to the formation of an active β-galactosidase enzyme, which then acts on substrate to emit light that can be detected as luminescence. These cell lines were thawed, grown, and maintained in Pathunter AssayComplete media (cat. no. 92-0018GF2).

Quantification of cAMP Levels

cAMP assays were optimized using PerkinElmer's LANCE ultra-cAMP kit (cat. no. TRF0262; PerkinElmer, Boston, Mass.) per the manufacturer's instructions. All assays were performed at room temperature using 384-optiplates (cat. no. 6007299; PerkinElmer). Briefly, cells were resuspended in 1× stimulation buffer (1× Hanks' balanced salt solution, 5 mM HEPES, 0.5 mM IBMX, 0.1% bovine serum albumin (BSA), pH 7.4, made fresh on the day of experiment). Cells (HEK CB2) were incubated for 1 hour at 37° C., 5% CO₂ and humidified air and then transferred to a 384-optiplate (500 cells/μ1, 10 μl), followed by stimulation with drugs/compounds and forskolin (2 μM final concentration) made in 1× stimulation buffer, as appropriate, for 5 minutes. For time-course experiments, cells were treated with CP55940 or LY282360 (in the presence of 2 μM forskolin final concentration) for defined times. For experiments with PTX, cells were treated overnight with 300 ng/ml PTX at 37° C. in 5% CO₂. Cells were then lysed by addition of 10 μl Eu-cAMP tracer working solution (4×, made fresh in 1× lysis buffer supplied with the kit, under subdued light conditions) and 10 μl Ulight anti-cAMP working solution (4×, made fresh in 1×lysis buffer) and further incubated for 1 hour at room temperature. Plates were then read with the TR FRET mode on an Enspire plate reader (PerkinElmer).

Detection of Phosphorylated ERK1/2

HEK-mCB2 or hCB2 were seeded on poly-D-lysine coated 96-well plates (75,000 cells/well) and grown overnight at 37° C., in 5% CO₂ humidified air. The following day, media was replaced by serum free DMEM, and plates were further incubated for 5 hours at 37° C. in 5% CO₂ humidified air. For experiments involving PTX, cells were treated overnight with PTX (300 ng/ml) and the next day serum-starved for 5 hours.

After serum starvation, the cells were challenged with drugs/compounds for the indicated time. After drug incubation, plates were emptied and quickly fixed with ice-cold 4% paraformaldehyde for 20 minutes, followed by ice-cold methanol with the plate maintained at −20° C. for 15 minutes. Plates were then washed with Tris-buffered saline (TBS)/0.1% Triton X-100 for 25 minutes (5×5-minute washes). The wash solution was then replaced by Odyssey blocking buffer and incubated further for 90 minutes with gentle shaking at room temperature. Blocking solution was then removed and replaced with blocking solution containing anti-phospho-ERK 1/2 antibody (1:150; Cell Signaling Technology, Danvers, Mass.) and was shaken overnight at 4° C. The next day, plates were washed with TBS containing 0.05% Tween-20 for 25 minutes (5×5-minute washes). Secondary antibody, donkey anti-rabbit conjugated with IR800 dye (Rockland, Limerick, Pa.), prepared in blocking solution, was added, and plates were gently shaken for 1 hour at room temperature. The plates were then again washed five times with TBS/0.05% Tween-20 solution. The plates were patted dry and scanned using LI-COR Odyssey scanner (LI-COR, Inc., Lincoln, Nebr.) phosphorylated ERK1/2 (pERK 1/2) activation (expressed in percentages) was calculated by dividing the average integrated intensities of the drug-treated wells by the average integrated intensities of vehicle-treated wells. All assays were performed in triplicate unless otherwise noted.

On-Cell Western for Receptor Internalization

HEK CB2 cells were grown to 95% confluence in DMEM+10% fetal bovine serum+0.5% Pen/Strep. Cells were washed once with HEPES-buffered saline/BSA (BSA @ 0.08 mg/ml) with 200 μl/well. Drugs were applied at the indicated concentrations to cells, after which they were incubated for 90 minutes at 37° C. Cells were then fixed with 4% paraformaldehyde for 20 minutes and washed four times (300 μl per well) with TBS. Blocking buffer (Odyssey blocking buffer; LI-COR, Inc., Lincoln, Nebr.) was applied at 100 μl per well for 1 hour at room temperature. Anti-hemagglutinin antibody (mouse monoclonal, 1:200; Covance, Princeton, N.J.) diluted in Odyssey blocking buffer was then applied for 1 hour at room temperature. After this, the plate was washed five times (300 μl/well) with TBS. Secondary antibody diluted (anti-mouse 680 antibody 1:800, LI-COR, Inc.,) in blocking buffer was then applied for 1 hour at room temperature, after which the plate was washed five times (300 μl/well) with TBS. The plate was imaged using an Odyssey scanner (700 channel, 5.5 intensity, LI-COR, Inc.).

Myo-Inositol Phosphate-1 (IP1) Accumulation Assay

Accumulation of myo-inositol phosphate-1 (IP1), a downstream metabolite of IP3, was measured by using IP-One HTRFkit (cat. no. 62, IPAPEB; Cisbio, Bedford, Mass.). Functional coupling of CB₂ receptor to G_{q} G protein leads to phospholipase Cβ (PLC) activation and initiation of the IP hydrolysis cascade. Accumulated IP3 is quickly dephosphorylated to IP2 and then to IP1. This assay takes advantage of the fact that accumulated IP1 is protected from further dephos-phorylation by the addition of lithium chloride, and IP1 levels can be easily quantified using an homogeneous time-resolved fluorescence (HTRF) assay. HEK mCB2 cells were detached from ˜50% confluent plates using versene. Cells (10 μl, 5000 cells) were resuspended in 1×stimulation buffer (containing lithium chloride, supplied with the kit) and were incubated for 1 hour at 37° C., 5% CO₂, and humidified air and then transferred to a 384-optiplate, followed by stimulation with drugs/compounds made in DMSO/ethanol as appropriate, for defined time points. Cells were then lysed with 5 μl of IP1-d2 dye (made fresh in lysis buffer, supplied with the kit), followed by the addition of 5 μl Ab-Cryptate dye (made fresh in lysis buffer). Plates were incubated further for 60 minutes at room temperature and then read in HTRF mode on an Enspire plate reader. All cell-based assay experiments were performed in triplicate unless otherwise stated.

General In Vivo Experimental Protocol

In all studies, the experimenter was blinded to the treatment condition, and mice were randomly assigned to experimental conditions. Paclitaxel (4 mg/kg i.p.) was administered four times on alternate days (cumulative dose, 16 mg/kg i.p.) to induce neuropathic pain as described previously by our group (Deng et al., 2015). Control mice received an equal volume of cremophor-vehicle. Development of paclitaxel-induced allodynia was assessed on day 0, 4, 7, 11, and 14.

Effects of pharmacologic manipulations were assessed at 30 minutes after drug administration during the maintenance phase of paclitaxel-induced neuropathy (i.e., beginning day 18-20 after initial paclitaxel injection).

In experiment 1, we assessed the dose response and time course of acute administration of LY2828360 on mechanical and cold allodynia in WT (C57BL/6J) mice treated with paclitaxel or its cremophor-based vehicle.

In experiments 2 and 3, pharmacologic manipulations were performed once daily for 12 consecutive days in each of the two phases of chronic treatment. Four days separated phase 1 and phase 2 chronic dosing in all studies comprising two phases of chronic dosing. Experiments 2 and 3 were performed concurrently using overlapping cohorts that were tested with a single vehicle (phase 1), vehicle (phase 2) group.

In experiment 2, we examined the antiallodynic efficacy of chronic systemic administration of LY2828360 (3 mg/kg per day i.p.×12 days) or vehicle administered during phase 1 using paclitaxcl-treated WT and CB₂KO mice. We then assessed the antiallodynic efficacy of chronic systemic administration of vehicle or morphine (10 mg/kg per day i.p.×12 days) administered during phase 2 in the same animals. Responsiveness to mechanical and cold stimulation was evaluated on treatment days 1, 4, 8, and 12 during phase 1 and on treatment days 16, 19, 23, and 27 during phase 2 (i.e., phase 2 started on day 16).

In experiment 3, we assessed the antiallodynic efficacy of chronic administration of LY2828360 (3 mg/kg per day i.p.×12 days in phase 2) or vehicle in paclitaxel-treated WT and CB₂KO mice that previously developed tolerance to morphine. To induce morphine tolerance, mice received repeated once daily injections of morphine (10 mg/kg per day i.p.×12 days) in phase 1 treatment; vehicle or LY2828360 (3 mg/kg per day i.p.×12 days) was administered chronically in phase 2.

In experiment 4, we evaluated the impact of coadministration of morphine (10 mg/kg i.p.×12 days) with a submaximal dose of LY 2828360 (0.1 mg/kg per day i.p.×12 days) in WT and CB₂ KO mice.

In experiment 5, we evaluated whether chronic administration of LY2828360 would attenuate morphine-dependent withdrawal symptoms that were precipitated using the opioid receptor antagonist naloxone. Alter the last injection of morphine (on day 28 for two-phase treatments, on day 13 for coadministration treatment), we challenged WT or CB₂KO mice from experiments 2, 3, and 4 with vehicle, followed 30 minutes later by naloxone (5 mg/kg i.p.) to precipitate opioid receptor-mediated withdrawal. Mice were video-recorded for subsequent scoring of withdrawal-like behaviors for a 30-minute interval after challenge with vehicle or naloxone.

Assessment of Mechanical Allodynia

Paw withdrawal thresholds (grams) to mechanical stimulation were measured in duplicate for each paw using an electronic von Frey anesthesiometer supplied with a 90-g probe (model Alemo 2390-5; IITC, Woodland Hills, Calif.) as described previously. Mice were placed on an elevated metal mesh table and allowed to habituate under individual, inverted plastic cages to the testing platform for at least 20 minutes until exploratory behavior had ceased. Alter the habituation period, a force was applied to the midplantar region of the hind paw with a semiflexible tip connected to the anesthesiometer. Mechanical stimulation was terminated when the animal withdrew its paw, and the value of the applied force was recorded in grams. Mechanical paw withdrawal thresholds were obtained in duplicate for each paw and are reported as the mean of duplicate determinations from each animal, averaged across animals, for each group.

Assessment of Cold Allodynia

Response time (seconds) spent attending to (i.e., elevating, licking, biting, or shaking) the paw stimulated with acetone (Sigma-Aldrich) was measured in triplicate for each paw to assess cold allodynia as previously published. An acetone bubble (approximately 5 to 6 p.1) formed at the end of a blunt 1-ml syringe hub was gently applied to the plantar surface of the hind paw. Care was taken not to apply mechanical stimulation to the hind paw with the syringe itself. The total time the animal spent attending to the acetone-stimulated paw (i.e., elevation, shaking, or licking) was recorded over 1 minute after acetone application. Acetone was applied three times to each paw with a 3-minute interval between applications. Values for each animal were calculated as the mean of six determinations of acetone responsiveness derived from each mouse.

Evaluation of Opioid Receptor-Mediated Withdrawal Symptoms

WT (C57BL/6J) mice and CB₂KO mice that received either vehicle or morphine (10 mg/kg per day, i.p.) or a combination of morphine with LY2828360 (10 mg/kg per day i.p. morphine coadministered with 0.1 mg/kg per day i.p. LY2828360) for 12 days were challenged with vehicle followed by naloxone (5 mg/kg i.p.) to induce opioid withdrawal beginning 30 minutes after the last injection of the test drugs. Mice were video-taped, and the number of jumps was scored in 5-minute intervals for a total observation period of 30 minutes after challenge with either saline or naloxone (5 mg/kg i.p.).

Statistical Analyses

Paw withdrawal thresholds (mechanical) and duration of acetone-evoked behavior (cold) were calculated for each paw and averaged. Analysis of variance for repeated measures was used to determine the time course of paclitaxel-induced mechanical and cold allodynia. One-way analysis of variance was used to identify the source of significant interactions at each time point and compare postinjection responses with baseline levels, followed by Bonferroni's post hoc tests (for comparisons between groups). Appropriate comparisons were also made using Bonferroni's post hoc tests or planned comparison t tests (unpaired or paired, as appropriate). All statistical analyses were performed using IBM-SPSS Statistics version 24.0 (SPSS Inc., an IBM company, Chicago, Ill.). P<0.05 was considered statistically significant. Sample size calculations and power analyses were performed using Statmate 2.0 for windows (Graphpad Prism Software, San Diego Calif., www.graphpad.com).

Illustrative embodiments of the compositions and methods of the present disclosure are provided herein by way of examples. While the concepts and technology of the present disclosure are susceptible to broad application, various modifications, and alternative forms, specific embodiments will be described here in detail. It should be understood, however, that there is no intent to limit the concepts of the present disclosure to the particular forms disclosed, but on the contrary, the intention is to cover all modifications, equivalents, and alternatives consistent with the present disclosure and the appended claims. The following experiments were used to determine the effect of different concentrations and/or timing of cannabinoid CB₂ receptor agonist compounds and compositions (e.g., LY2828360).

### Example 1: LY2828360 Displays a Delayed, G Protein-Biased Signaling Profile at Mouse CB₂ Receptors

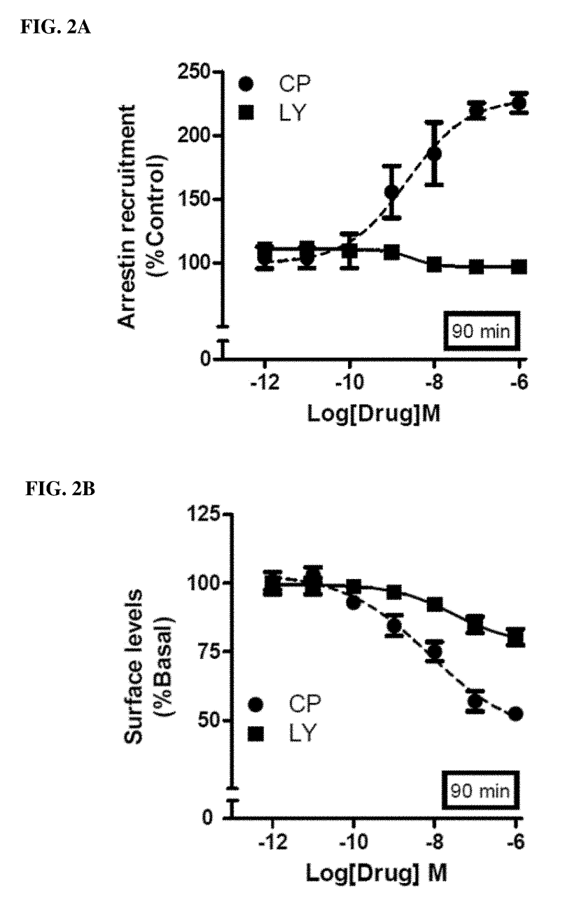

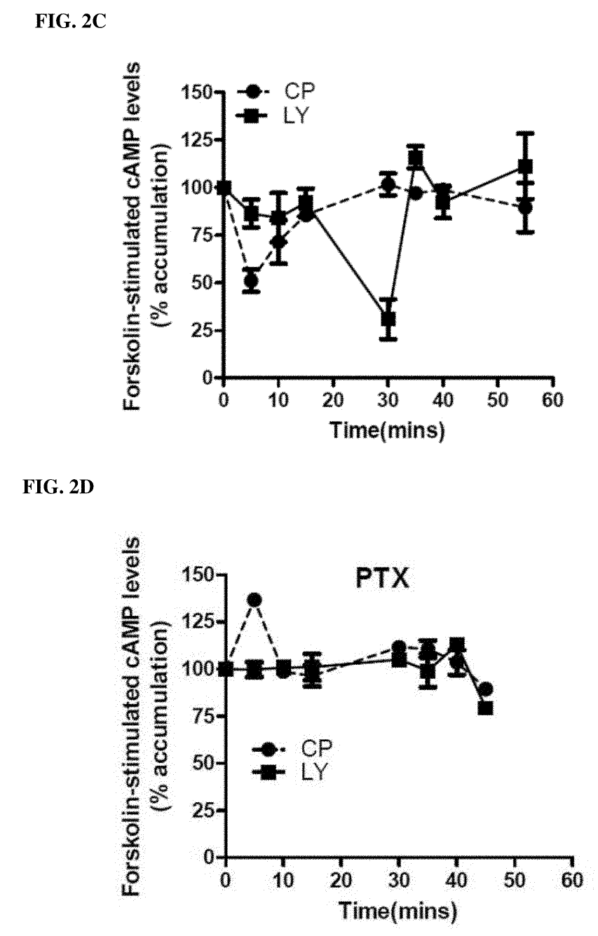

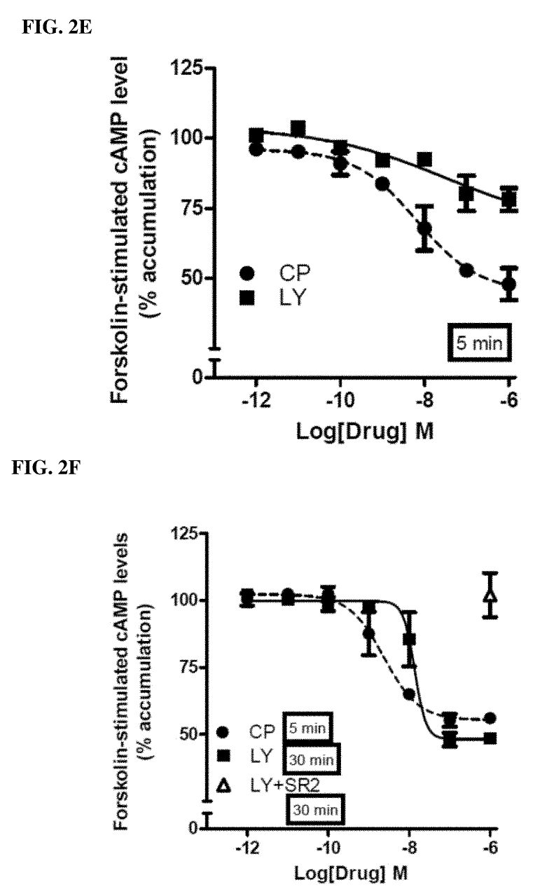

A range of cell-based in vitro signaling assays were vised to dissect the signaling of LY2828360 at CB₂ receptors. In an arrestin recruitment assay evaluating mouse CB₂ receptors, CP55940 recruited arrestin in a concentration-dependent manner, whereas LY2828360 failed to do so after a 90-minute drug incubation (FIG. 2A). Recruitment of arrestin is necessary for many forms of receptor sequestration and internalization. In congruency, LY2828360 failed to internalize the receptor (FIG. 2B). Strikingly, CP55940 (1 μM) induced a rapid (˜5 minutes) and efficacious inhibition of forskolin-stimulated adenylyl cyclase, and LY2828360 (1 μM) induced an efficacious inhibition only after 30 minutes (FIG. 2C). CB₂ receptor inhibition of adenylyl cyclase is mediated by inhibitory Gi/o G proteins.

Thus, to confirm whether delayed inhibition by LY2828360 was mediated by Gi/o G proteins, cells were pretreated with pertussis toxin (PTX), 300 ng/ml, overnight). After PTX treatment, LY2828360 no longer inhibited cAMP accumulation at 30 minutes (FIG. 2D), confirming involvement of inhibitory G proteins. Next, full-concentration response experiments were performed two times when maximal inhibition of forskolin-stimulated cAMP accumulation was observed. At 5 minutes, CP55940 potently and efficaciously inhibited cAMP accumulation, whereas LY2828360 had no effect (FIG. 2E; Table 1). Conversely, at 30 minutes, LY2828360 was potent, efficacious, and CB₂ receptor mediated (FIG. 2F).

More specifically, LY2828360 displays a delayed signaling profile at mouse CB₂ receptors. FIG. 2A demonstrates that in CHO cells stably expressing mCB₂ receptors, CP55940 recruited arrestin in a concentration-dependent manner, whereas LY2828360 failed to do so after 90-minute drug incubation. FIG. 2B shows that in HEK cells stably transfected with mCB2, CP55940 concentration dependently internalized the mCB2; LY2828360 was less potent and efficacious. FIG. 2C demonstrates that in a forskolin-stimulated cAMP time course assay, CP55940 (1 μM) was efficacious and rapid in inhibiting forskolin-stimulated cAMP accumulation at 5 minutes, whereas LY2828360 (1 μM) was efficacious only after 30 minutes. FIG. 2D shows that after PTX treatment, CP55940 (1 μM) modestly increased cAMP accumulation at 5 minutes, whereas LY2828360 (1 μM) failed to affect cyclase levels at all time points examined/tested. FIG. 2E demonstrates that CP55940 was potent and efficacious in inhibiting forskolin-stimulated cAMP accumulation at 5 minutes, whereas LY2828360 failed to affect cAMP levels at this time point. FIG. 2E shows that after 30-minute incubation, however, LY2828360 concentration dependently inhibited forskolin-stimulated cAMP accumulation, and this inhibition was completely blocked by 1 μM SR144528 (SR2). Forskolin-stimulated cAMP assays were performed in duplicate. All other assays were performed in triplicate. All data were plotted and analyzed using GraphPad Prism 4.

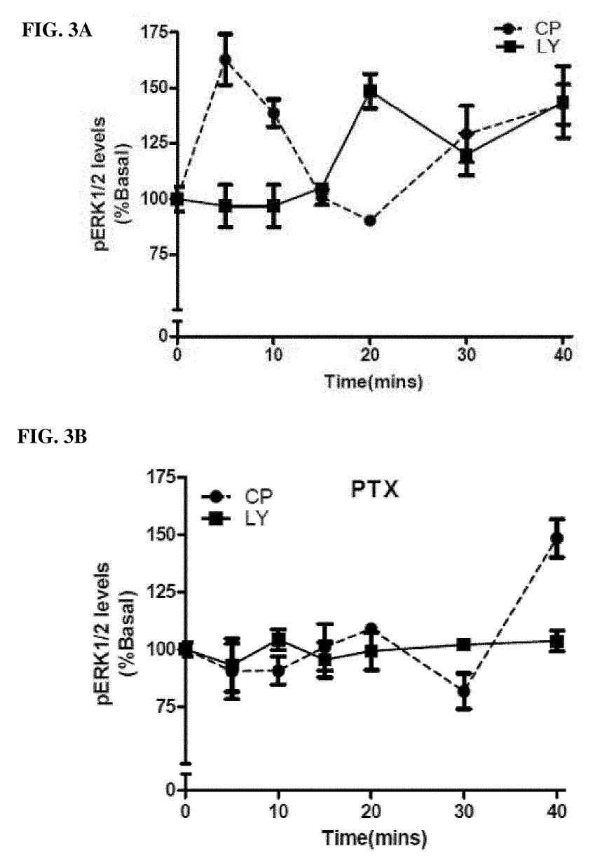

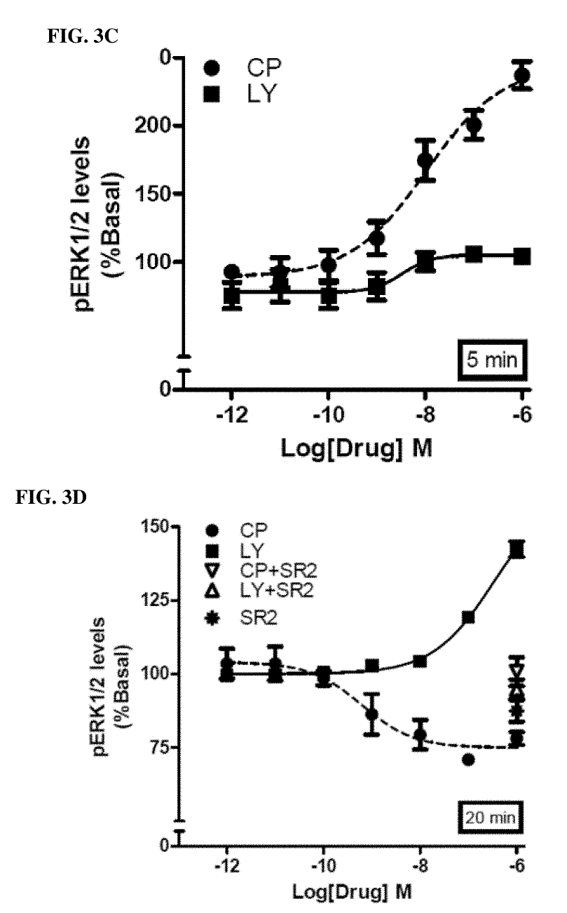

CP55940 (1 μM) was efficacious in stimulating ERK1/2 phosphorylation (pERK 1/2) at 5, 10, 30, and 40 minutes. On the other hand, LY2828360 (1 μM) increased pERK1/2 only at later times, such as at 20, 30, and 40 minutes. ERK1/2 activation by LY2828360 was completely abolished by pretreatment of cells with PTX (300 ng/ml; overnight) (FIGS. 3A and 3B), demonstrating G protein dependence. In contrast, only the early phase of CP55940 stimulation of pERK 1/2 was PTX sensitive, consistent with the delayed phase of pERK 1/2 activation by CP55940 being arrestin-mediated. A full concentration response experiment revealed that LY2828360 failed to increase pERK1/2 at 5 minutes but was potent and efficacious at 20 minutes and required CB₂ receptors as it was blocked by SR144528 (FIGS. 3C and 3D; Table 1).

LY282360 displays a delayed CB₂ receptor- and G protein-dependent signaling profile in activating pERK1/2. FIG. 3A demonstrates that in HEK cells stably expressing mouse CB2 receptors, CP55940 (1 μM) increased phosphorylated ERK1/2 at 5-, 10-, 30-, and 40-minute time points, whereas LY2828360 (1 μM) had no effect at 5- and 10-minute time points but increased ERK1/2 phosphorylation at 20, 30, and 40 minutes. FIG. 3B shows that PTX treatment abolished the 20-minute phosphorylation of ERK1/2 by LY2828360 (1 μM) and abolished the CP55940-mediated phosphorylation of ERK1/2 at the 5-minute time point, but it was retained at the 40-minute time point after PTX treatment. FIG. 3C demonstrates that CP55940 concentration dependency increased ERK1/2 phosphorylation at 5 minutes, whereas LY2828360 failed to affect pERK1/2 levels at this time point. (D) Conversely, FIG. 3D shows that after 20 minutes of treatment, CP55940 decreased ERK1/2 phosphorylation, whereas LY2828360 increased ERK1/2 phosphorylation, in a concentration-dependent manner. Both effects were blocked by the CB₂ receptor antagonist SR144528 (1 μM) (SR2).

pERK 1/2 assays were performed in triplicate. All the experimental data were plotted and analyzed using GraphPad Prism4. Potencies and efficacies of CP55940 and LY2828360 in the signaling assays described at mouse and human CB₂ receptors are summarized in Tables 1 and 2, respectively (below).

### Example 2: Effects of Acute Administration of LY2828360 in Paclitaxel-Treated WT Mice

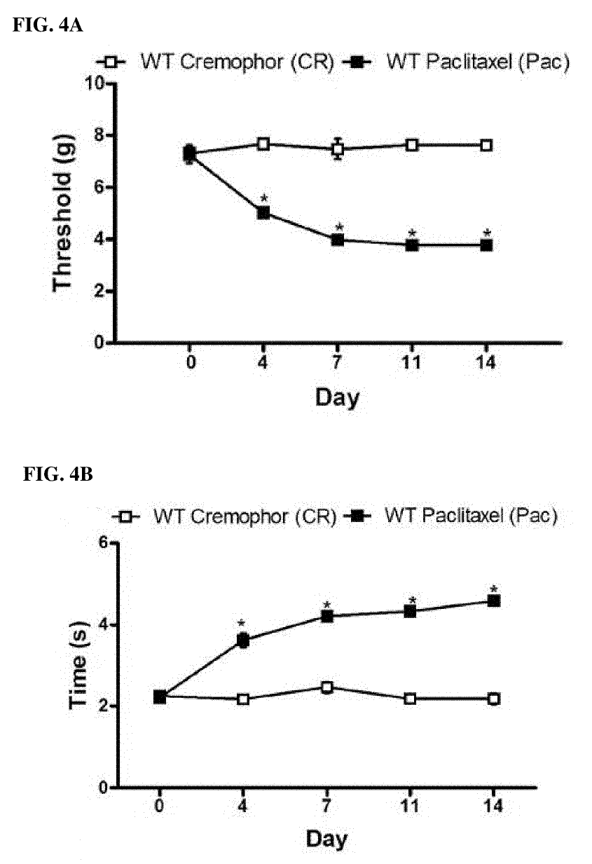

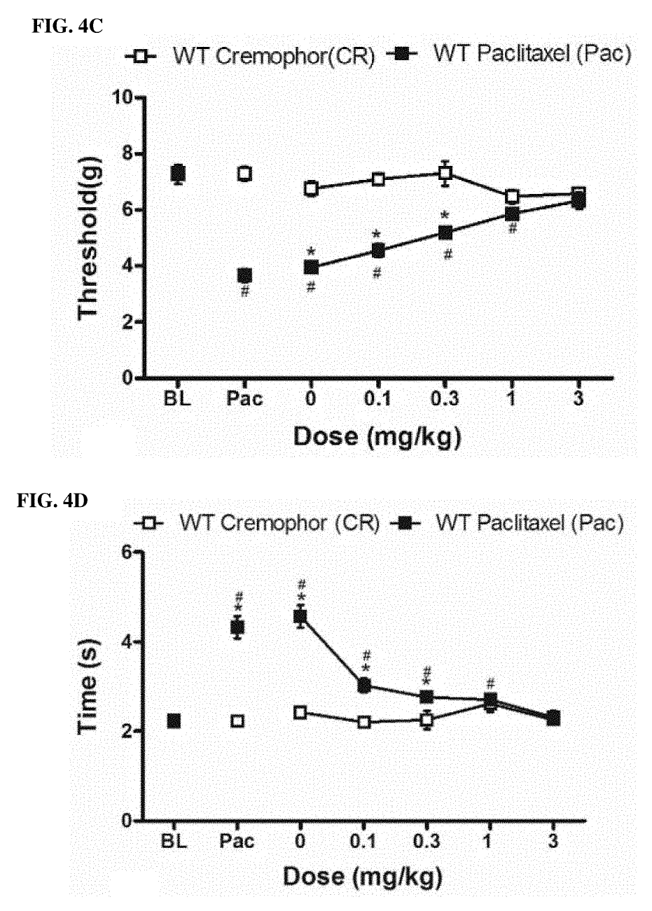

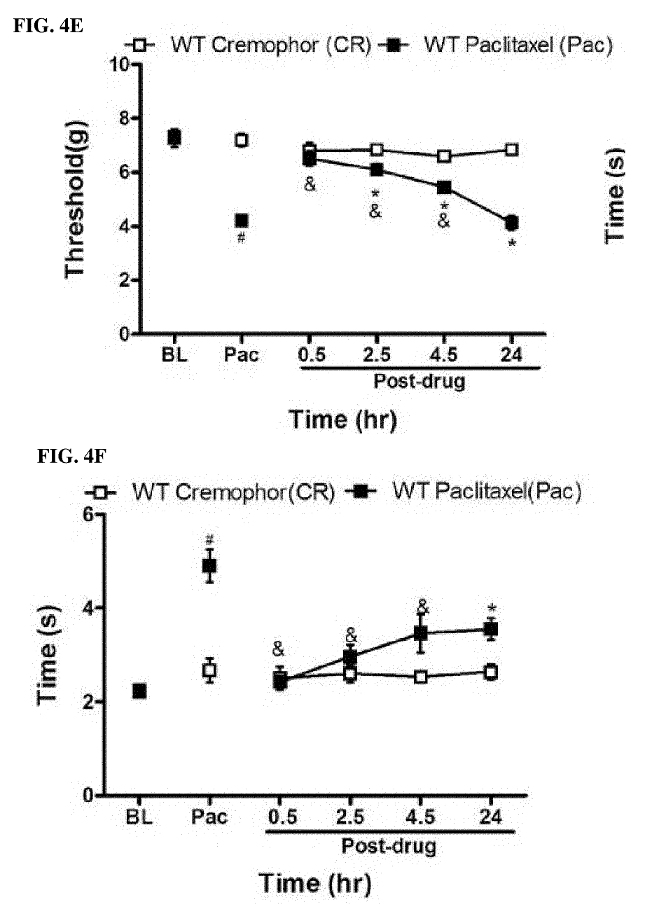

Paclitaxel decreased paw-withdrawal thresholds (F)_{1,10}=249.98, P=0.0001) and increased acetone-evoked behaviors (F)_{1,10}=342.95, P=0.0001), consistent with our previous studies showing development of mechanical and cold allodynia after paclitaxel treatment in mice. Thus, mechanical (FIG. 4A) and cold (FIG. 4B) allodynia developed by day 4 (P=0.0001) after initial paclitaxel dosing and was maintained with high stability in paclitaxel-treated WT mice relative to cremophor-vehicle treatment from day 7 onward (P=0.0001).

In WT mice, acute systemic administration of LY2828360 suppressed paclitaxel-induced mechanical (F_{1,10}=125.902, P=0.0001; FIG. 4C) and cold (F_{1,10}=29.167, P=0.0001; FIG. 4D) allodynia in a dose-dependent manner. The high dose of LY2828360 (3 mg/kg i.p.) fully reversed paclitaxel-induced allodynia and normalized responses to pre-paclitaxel baseline levels (P=0.167 mechanical; P=0.53 cold) (FIGS. 4C and 4D); however, neuropathic pain was prominent in paclitaxel-treated mice receiving doses of LY2828360 lower than 0.3 mg/kg i.p. compared with control mice that received the cremophor-vehicle in lieu of paclitaxel (P=0.001 mechanical; P=0.044 cold).

To study the duration of antinociceptive action of LY2828360, the maximally efficacious dose (3 mg/kg i.p.) was administered to paclitaxel-treated mice and responsiveness to mechanical and cold stimulation was evaluated at 0.5, 2.5, 4.5, and 24 hours postinjection. LY2828360 produced time-dependent suppressions of paclitaxel-evoked mechanical (F_{1,10}=38.604 P=0.0001; FIG. 4E) and cold (F_{1,10}=4.993, P<0.05 cold; FIG. 4F) hypersensitivities and suppression of allodynia was maintained for at least 4.5 hours postinjection (P=0.001 mechanical, P=0.022 cold) relative to drug reinjection levels (i.e., Pac). At 24 hours postinjection, paclitaxel-induced mechanical allodynia had returned (P=1 mechanical; P=0.125 cold) to drug preinjection levels of hypersensitivity (FIGS. 4E and 4F). Residual suppression of cold allodynia was absent by 72 hours after LY2828360 treatment (data not shown).

Paclitaxel produced hypersensitivities to mechanical (FIG. 4A) and cold (FIG. 4B) stimulation. Non-chemotherapy control mice received cremophor-based vehicle in lieu of paclitaxel. Dose response of LY2828360, administered systemically (i.p.), on the maintenance of mechanical (FIG. 4C) and cold (FIG. 4D) allodynia in paclitaxel-treated WT (C57BL/6J) mice.

The time course of LY2828360, administered systemically (3 mg/kg i.p.), on the maintenance of mechanical (FIG. 4E) and cold (FIG. 4F) allodynia in paclitaxel-treated WT mice. Data are expressed as mean±S.E.M. (n=6/group). *P<0.05 vs. control, one-way analysis of variance at each time point, followed by Bonferroni's post hoc test. #P<0.05 vs. baseline before paclitaxel, repeated measures analysis of variance. ^{&}P<0.05 vs. baseline after paclitaxel, repeated measures analysis of variance. BL, pre-paclitaxel baseline; Pac, baseline after paclitaxel.

TABLE 1

| | Drug | CP55940 | | | | LY2828360 | | | |
|---|---|---|---|---|---|---|---|---|---|
| | incubation | EC50 | | Emax | | EC50 | | Emax | |
| | (mins) | (nM) | 95% CI | (%) | ±SEM | (nM) | 95% CI | (%) | ±SEM |
| Arrestin | 90 | 2.3 | 0.4-12.2 | 125 | ±1.6 | ND | ND | 97.9* | ±1.5 |
| Internalization | 90 | 7.4 | 1.1-19.3 | 49.1 | ±1.2 | 30.7 | 1.4-626.5 | 19.1 | ±2.4 |
| Cyclase | 05 | 6.6 | 1.7-12.2 | 52.8 | ±3.6 | ND | ND | 18.9 | ±5.8 |
| | 30 | — | — | — | — | 13.6 | 10.4-45.3 | 53.4 | ±1.9 |
| pERK1/2 | 05 | 10.5 | 2.2-17.9 | 136.2 | ±4.1 | ND | ND | 4.1 | ±2.5 |
| | 20 | 1.5 | 0.1-3.7 | 20.3* | ±3.4 | 339 | 128.8-345.8 | 43.6 | ±2.3 |

TABLE 2

| | Drug | CP55940 | | | | LY2828360 | | | |
|---|---|---|---|---|---|---|---|---|---|
| | incubation | EC50 | | Emax | | EC50 | | Emax | |
| | (mins) | (nM) | 95% CI | (%) | ±SEM | (nM) | 95% CI | (%) | ±SEM |
| Internalization | 90 | 3 | 0.3-15.6 | 33.9 | ±4.6 | ND | ND | 10.2 | ±7.1 |
| Cyclase | 05 | 12.3 | 2.9-18.3 | 59.6 | ±8.3 | ND | ND | ND | ND |
| | 35 | — | — | — | — | 16.7 | 4.6-59.6 | 42.8 | ±2.7 |
| pERK1/2 | 05 | 3.77 | 0.4-12.7 | 95.7 | ±9.1 | ND | ND | 22.1* | ±5.8 |
| | 30 | 23.3 | 10.1-53.9 | 49.4 | ±1.6 | 33.5 | 9.1-107.1 | 32.3 | ±1.9 |

### Example 3: Previously Chronic Administration of LY2828360 Blocked the Development of Tolerance to the Anti-Allodynic Effects of Morphine in WT but not in CB2KO Mice

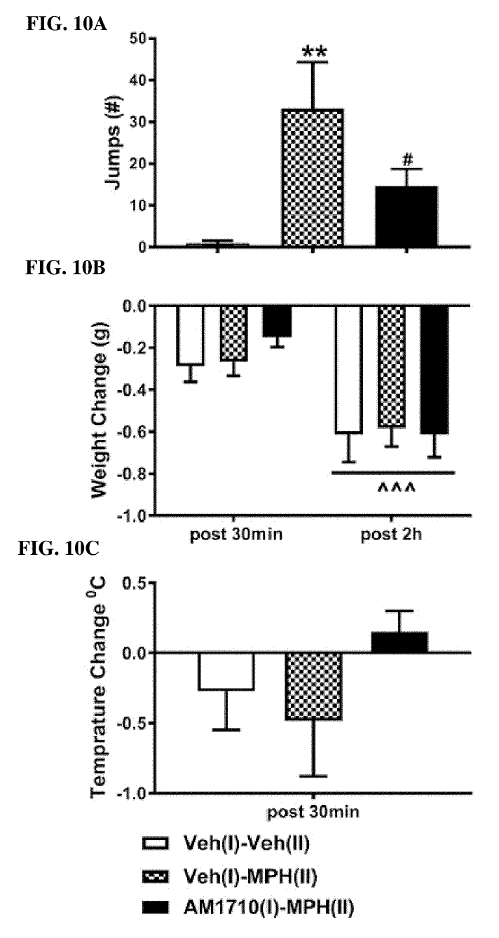

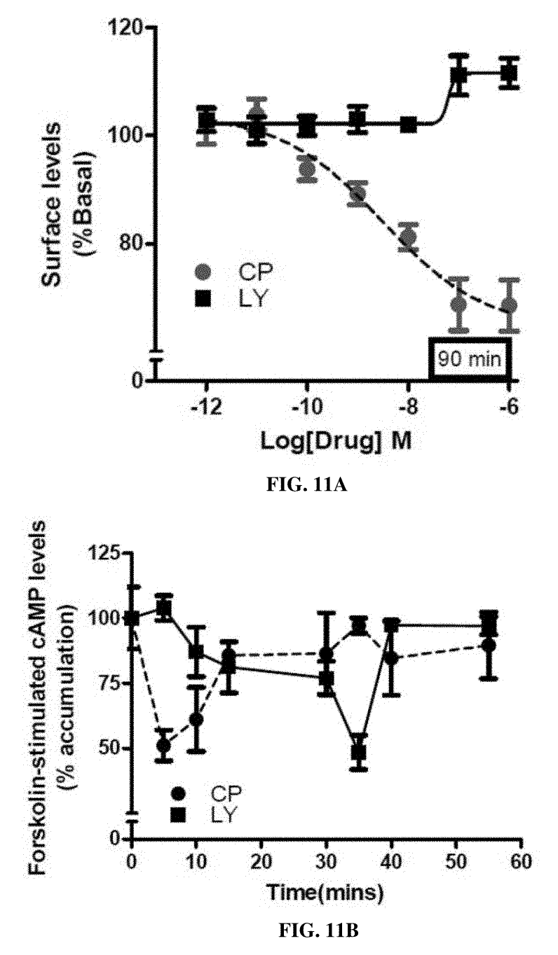

To study the effects of LY2828360 treatment on the development of tolerance to morphine, pharmacologic manipulations were used in two phases of treatment during the maintenance of neuropathic pain (FIG. 10A). In Wildtype (WT) mice, phase 1 treatment with LY2828360 (3 mg/kg per day i.p.×12 days) suppressed paclitaxel-induced mechanical (F_{2,15}=183.929, P=0.0001; FIG. 10B) and cold (F_{2,15}=64.218, P=0.0001; FIG. 10C) hypersensitivities relative to phase 1 vehicle treatments.

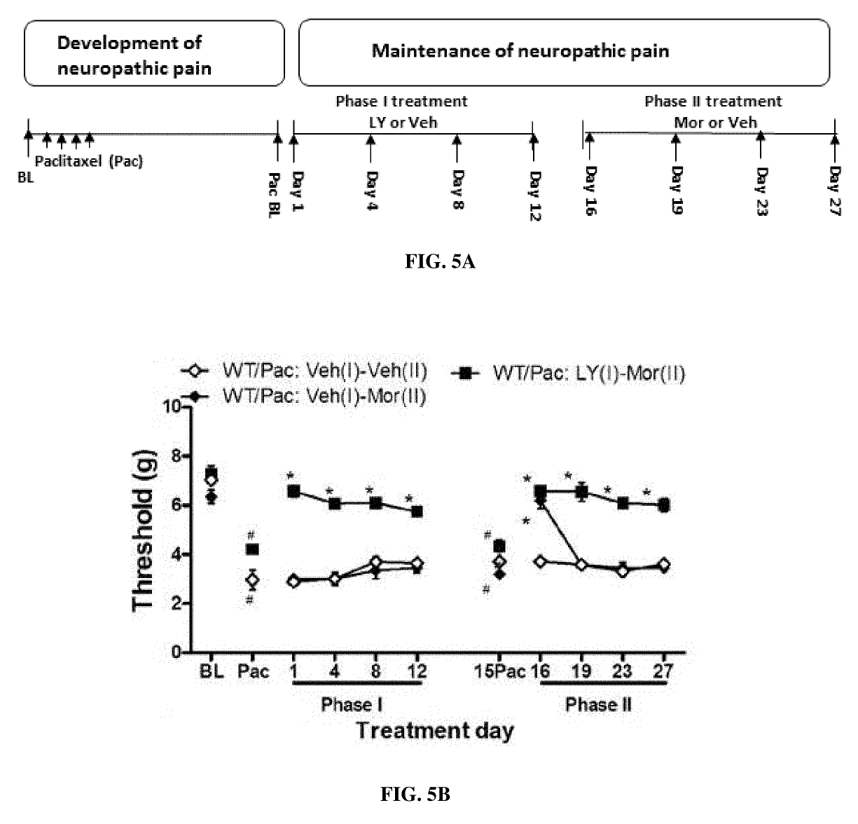

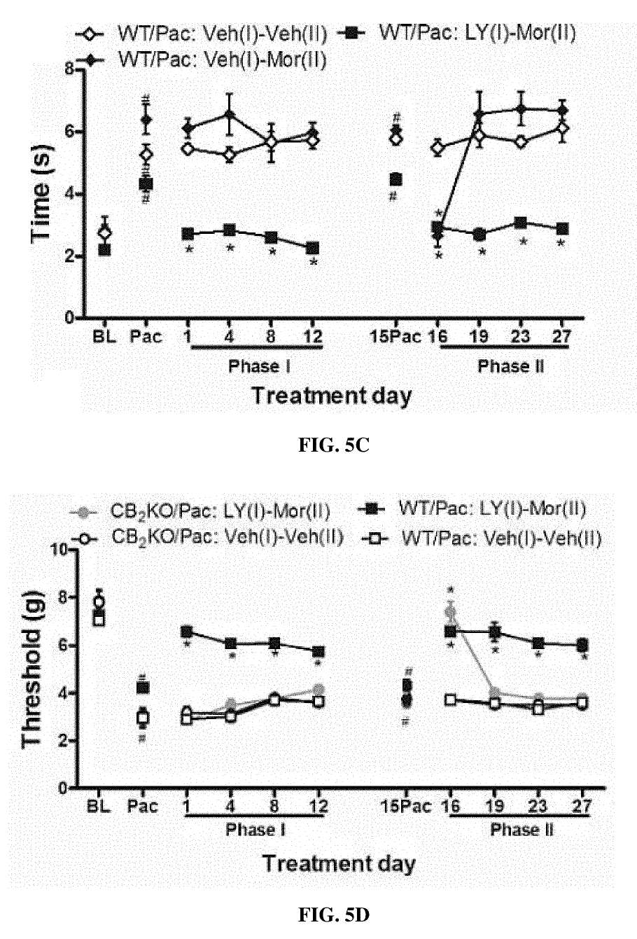

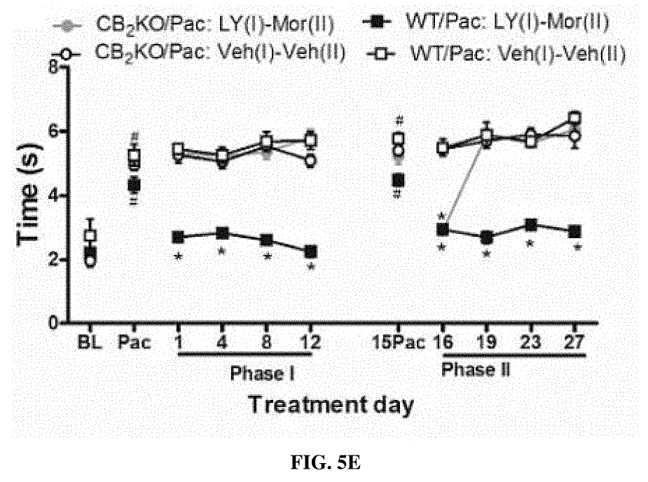

LY2828360 markedly suppressed paclitaxel-induced mechanical and cold allodynia throughout the observation interval (P=0.0001 mechanical; P=0.016 cold; FIGS. 5B and 5C). Mechanical and cold hypersensitivities were largely normalized by LY2828360 (3 mg/kg i.p.×12 days) with responses returning to baseline (i.e., pre-paclitaxel) levels (P=0.138 mechanical; P=0.182 cold). The antiallodynic efficacy of LY2828360 was stable throughout phase 1 treatment (P=0.310 mechanical, P=0.314 cold) without the development of tolerance (FIGS. 5B and 5C).

On day 15, 3 days after the completion of phase 1 treatment, paclitaxel-induced mechanical and cold allodynia had returned to levels comparable to those observed before the initiation of phase 1 treatment (i.e., Pac; P=0.379 mechanical, P=0.62 cold; FIGS. 5B and 5C). Mechanical and cold allodynia were maintained in these mice relative to pre-paclitaxel levels (i.e., baseline; P<0.005 mechanical, P<0.006 cold). In paclitaxel-treated WT mice, chronic morphine treatment during phase 2 of mice previously receiving vehicle during phase 1 [WT/Pac: Veh (vehicle) (1)-Mor (morphine) (2)] only suppressed paclitaxel-induced mechanical and cold allodynia on day 16 (P=0.0001 mechanical, P=0.0001 cold) and then failed to suppress paclitaxel-induced mechanical (P=1) and cold (P=1) allodynia on subsequent test days (i.e., days 19, 23, and 27) relative to vehicle-treated mice [WT/Pac: Veh (1)-Veh (2); FIGS. 5B and 5C]. Thus, morphine tolerance rapidly developed to the antiallodynic effects of phase 2 morphine in paclitaxel-treated mice receiving vehicle in phase 1.

By contrast, in WT mice receiving LY2828360 during phase 1, phase 2 morphine [WT/Pac: LY (1>Mor (2); 10 mg/kg i.p.×12 days] sustainably suppressed paclitaxel-induced mechanical (F_{2,15}=91.428, P=0.0001) (FIG. 10B) and cold (F_{2,15}=40.979, P=0.0001; FIG. 10C) hypersensitivities relative to mice pretreated with vehicle in phase 1 [WT/Pac: Veh (1)-Mor (2); P=0.0001] (FIGS. 5B and 5C). This suppression was present and stable throughout phase 2 for both mechanical (P<0.05) and cold (P<0.009) modalities compared with drug preinjection levels in phase 2 (i.e., day 15). Morphine-induced antiallodynic efficacy was stably maintained throughout the observation interval after LY2828360 pretreatment for each stimulus modality (P=0.222 mechanical, P=0.535 cold). Thus, a previous history of chronic treatment with LY2828360 prevented the development of morphine tolerance in paclitaxel-treated WT mice for both stimulus modalities.

In paclitaxel-treated CB₂KO mice, phase 1 LY2828360 (3 mg/kg per day i.p.×12 days) treatment failed to suppress mechanical (P>0.05) or cold (P>0.05) allodynia relative to vehicle treatment on any day (FIGS. 5D and 5E). In these same CB₂KO mice, subsequent phase 2 morphine treatment [CB₂KO/Pac: LY (1)-Mor (2)] suppressed only mechanical (P=0.0001) and cold (P=0.0001) allodynia on the initial day of morphine dosing (i.e., day 16) relative to vehicle treatment [CB₂KO/Pac: Veh (1)-Veh (2)]. Paclitaxel-induced allodynia was fully reinstated at subsequent time points (i.e., on days 19, 23, and 27; P=1 mechanical, P=0.269 cold). The antiallodynic efficacy of initial morphine administration (i.e., on day 16) was similar in WT mice and CB₂KO mice (P=0.203 mechanical; P=1 cold). Phase 2 morphine administration continued to suppress paclitaxel-induced allodynia (P=0.0001 mechanical; P=0.0001 cold) in WT mice previously receiving LY2828360 [WT/Pac: LY (1)-Mor (2)] but not in the CB₂KO mice at subsequent time points (i.e., days 19, 23, and 27), suggesting that pretreatment with LY2828360 did not block the development of morphine tolerance in CB₂KO mice.

History of chronic LY2828360 treatment blocked the development of morphine tolerance in WT but not in CB2KO mice. FIG. 10A shows the testing scheme used to evaluate the two phases of treatment during the maintenance of neuropathic pain. History of chronic LY2828360 (3 mg/kg per day i.p.×12 days in phase 1) treatment suppressed paclitaxel-induced mechanical (FIG. 10B) cold (FIG. 10C) allodynia in WT mice.

History of chronic LY2828360 (3 mg/kg per day i.p.×12 days in phase 1) blocked the development of tolerance to the antiallodynic effects of morphine (10 mg/kg per day×12 days in phase 2) in WT but not in CB₂K0 mice for both mechanical (FIG. 10D) and cold (FIG. 10E) modalities. Data are expressed as mean±S.E.M. (n=6/group). *P<0.05 versus Veh (1)-Veh (2), oneway analysis of variance at each time point, followed by Bonferroni's post hoc test. ″P<0.05 vs. baseline before paclitaxel, repeated measures analysis of variance.

### Example 4: Chronic LY2828360 Treatment Suppresses Paclitaxel-Induced Mechanical and Cold Allodynia in WT Mice but not in CB₂KO Mice Previously Rendered Tolerant to Morphine

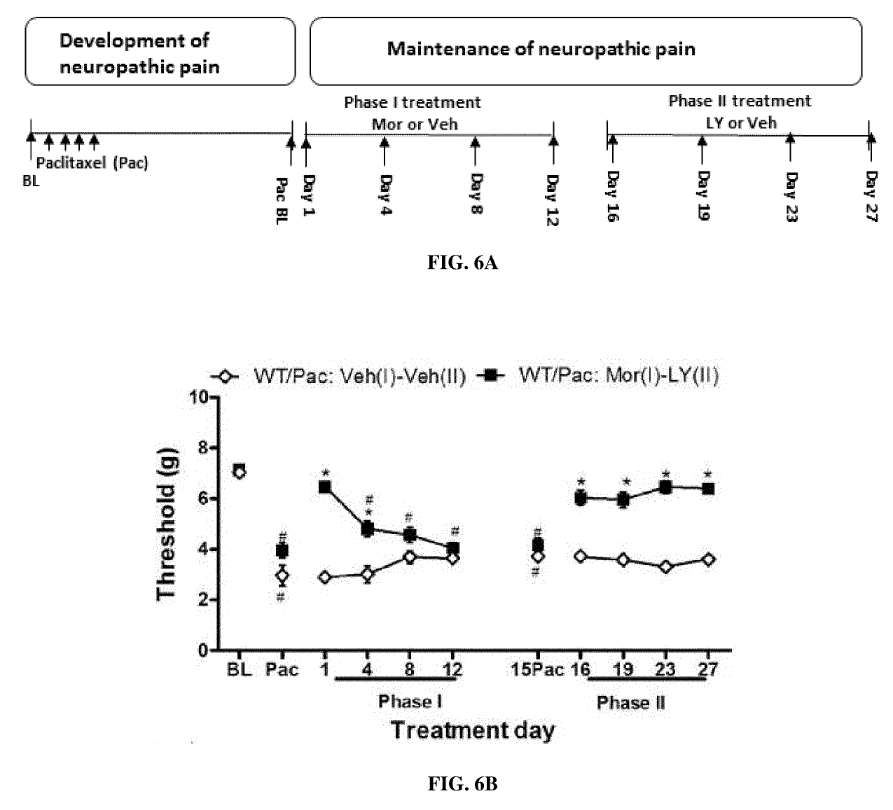

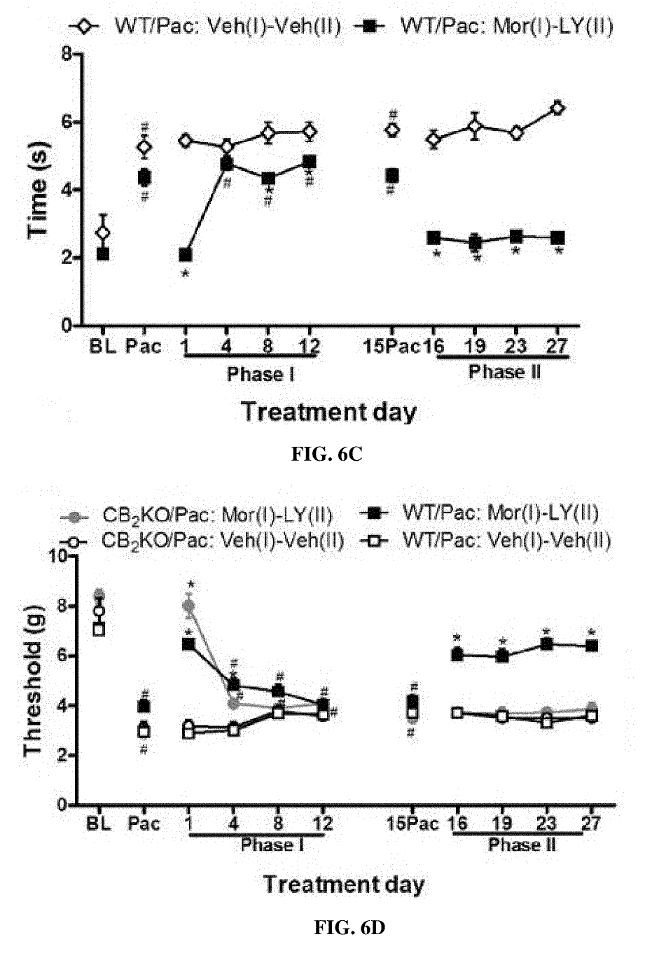

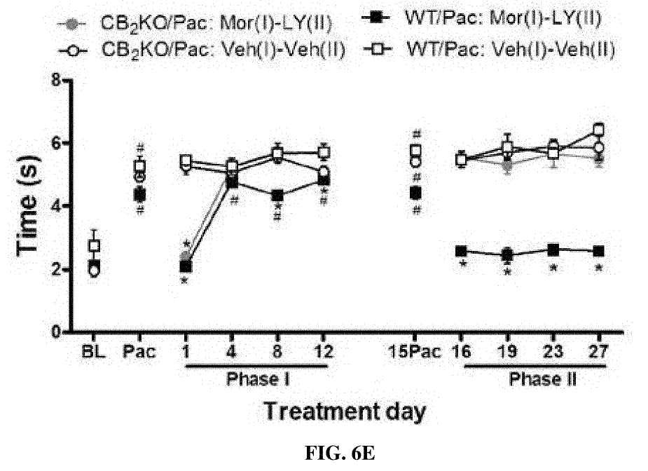

To evaluate whether LY2828360 has antiallodynic efficacy in morphine-tolerant mice, we first dosed paclitaxel-treated WT and CB₂KO mice chronically with morphine during phase 1 (10 mg/kg per day i.p.×12 days) and continued with chronic LY2828360 administration (3 mg/kg per day i.p.×12 days) (FIG. 6A) in phase 2. In phase 1, morphine administration suppressed paclitaxel-induced mechanical (F_{1,10}=83.817 P=0.0001) and cold (F_{1,10}=99.443, P=0.0001) allodynia relative to vehicle treatment. On day 1, morphine fully reversed paclitaxel-induced allodynia and normalized responses to pre-paclitaxel levels (i.e., baseline; P=0.062 mechanical; P=1.0 cold) but not on subsequent test days (i.e., day 4, 8, 12; FIGS. 6B and 6C). Antiallodynic efficacy of morphine was decreased on subsequent test days relative to pre-paclitaxel levels of responsiveness (P=0.005 mechanical; P=0.0001 cold). Thus, tolerance developed to the antiallodynic effects of morphine (i.e., on day 4, 8 and 12) (FIGS. 6B and 6C).

To evaluate whether LY2828360 produces antiallodynic effects in mice previously rendered tolerant to morphine, LY2828360 (3 mg/kg per day i.p.×12 days) was administered during phase 2 to paclitaxel-treated mice that previously receiving morphine during phase 1. Phase 2 LY2828360 (3 mg/kg per day i.p.×12 days) treatment fully reversed paclitaxel-induced allodynia and normalized responsiveness to pre-paclitaxel baseline levels in WT mice that previously developed morphine tolerance in phase 1 (P=0.112 mechanical; P=0.103 cold; FIGS. 6B and 6C). Thus, prior morphine tolerance does not attenuate LY2828360-induced antiallodynic efficacy in phase 2 in WT mice. Antiallodynic efficacy of LY2828360 was also stable throughout the chronic dosing period (P=1.0 mechanical; P=1.0 cold), suggesting that tolerance did not develop to phase 2 LY2828360 treatment in WT mice (FIGS. 6B and 6C).

To further evaluate the mechanism of action underlying the antiallodynic efficacy of LY2828360, we compared the efficacy of phase 2 LY2828360 treatment in CB₂KO and WT mice that were rendered tolerant to morphine during phase 1. Acute morphine increased paw withdrawal thresholds and reduced cold response times in paclitaxel-treated CB₂KO mice relative to the vehicle treatment on day 1 of phase 1 dosing (P=0.0001 mechanical; P=0.0001 cold) (FIGS. 6D and 6E). The antiallodynic effects of phase 1 morphine were attenuated on day 4 (P=0.058 mechanical; P=0.992 cold) and morphine antiallodynic efficacy was completely absent on day 8 and day 12 of chronic dosing (P=1.0 mechanical; P=1.0 cold; FIGS. 6D and 6E).

Chronic administration of LY2828360 in phase 2 (3 mg/kg per day, i.p.×12 days) did not alter responsiveness to mechanical or cold stimulation in paclitaxel-treated CB₂KO mice relative to the vehicle treatment at any time point (P=0.252 mechanical; P=0.299 cold) (FIGS. 6D and 6E). Thus, chronic administration of LY2828360 produced antiallodynic efficacy in paclitaxel-treated WT mice but not CB₂KO with the same histories of morphine treatment (P=0.0001 mechanical, P=0.0001 cold). In fact, Chronic LY2828360 treatment showed sustained antiallodynic efficacy in morphine-tolerant WT mice but not in CB2KO mice.

FIG. 6A shows a testing scheme used to evaluate the two phases of treatment during the maintenance of neuropathic pain. Chronic LY2828360 (3 mg/kg per day i.p.×12 days in phase 2) treatment suppressed paclitaxel-induced mechanical (FIGS. 6A and 6D) and cold (FIGS. 6C and 6E) allodynia in WT mice but not in CB₂KO mice previously rendered tolerant to morphine (10 mg/kg per day i.p.×12 days in phase 1). Data are expressed as mean±S.E.M. (n=6/group). Veh (1)-Veh (2) group is replotted from FIG. 10. *P<0.05 vs. Veh (1)-Veh (2), one-way analysis of variance at each time point, followed by Bonferroni's post hoc test. ^{#}P<0.05 vs. baseline before paclitaxel, repeated measures analysis of variance.

### Example 5: Chronic Coadministration of Low-Dose LY2828360 with Morphine Blocked Morphine Tolerance in WT but not in CB₂ KO Mice

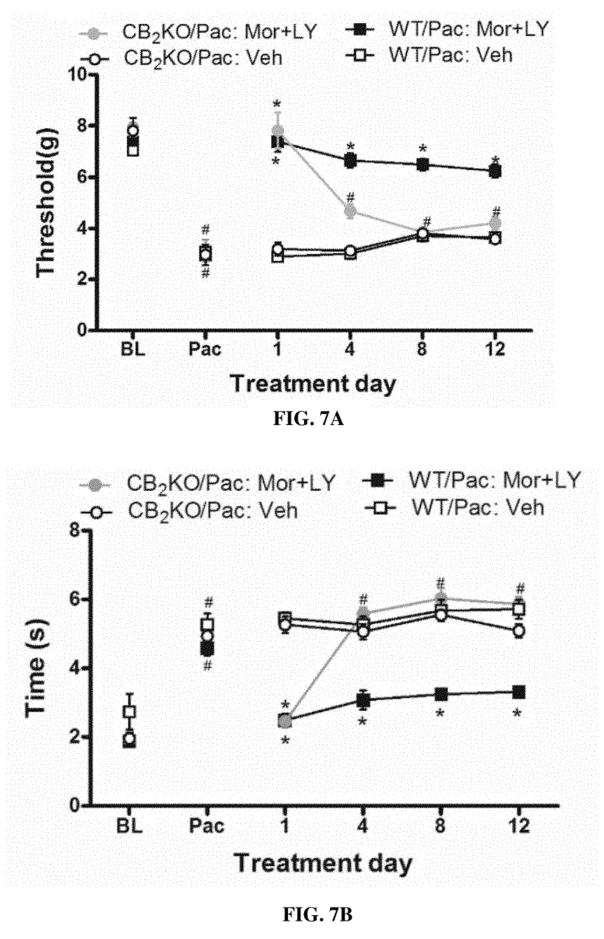

In WT mice, coadministration of a submaximal dose of LY2828360 (0.1 mg/kg per day i.p.×12 days) with morphine (10 mg/kg per day×12 days) suppressed paclitaxel-induced mechanical (F_{3,20}=111.039 P=0.0001) (FIG. 7A) and cold (F_{3,20}=56.823 P=0.0001; FIG. 7B) hypersensitivities relative to vehicle treatment (P=0.0001). Coadministration of the CB₂ agonist with morphine fully reversed paclitaxel-induced mechanical allodynia and normalized responses to pre-paclitaxel baseline levels throughout the observation period (P=0.078).

Coadministration of the CB₂ agonist with morphine also normalized cold responsiveness on days 1 and 4 (P=0.156) of chronic dosing to pre-paclitaxel baseline levels. By contrast, in CB₂KO mice, sustained antiallodynic efficacy was absent in paclitaxel-treated mice receiving LY2828360 co-administered with morphine; the combination treatment reversed only paclitaxel-induced mechanical (P=0.0001) and cold (P=0.0001) allodynia relative to vehicle on day 1 (FIGS. 7A and 7B).

Antiallodynic efficacy of morphine co-administered with LY2828360 was greater in WT mice relative to CB₂KO mice on subsequent days of chronic dosing (i.e., days 4, 8, and 12; P=0.0001 mechanical; P=0.0001 cold) (FIGS. 7A and 7B). In paclitaxel-treated WT mice, the combination of morphine with LY2828360 produced a stable, sustained antiallodynic efficacy throughout the dosing period (P=0.344 mechanical; P=0.995 cold), demonstrating that morphine tolerance failed to develop in the coadministration condition (FIGS. 7A and 7B).

Chronic coadministration of low-dose LY2828360 (0.1 mg/kg per day i.p.×12 days) with morphine (10 mg/kg per day i.p.×12 days) blocked development of morphine tolerance in WT but not in CB₂KO mice tested for both mechanical (FIG. 7A) and cold (FIG. 7B) allodynia. Data are expressed as mean±S.E.M. (n=6/group). *P<0.05 vs. WT-Veh, one-way analysis of variance at each time point, followed by Bonferroni's post hoc test. ^{#}P<0.05 vs. baseline before paclitaxel, repeated measures analysis of variance.

### Example 6: Naloxone-Precipitated Withdrawal is Attenuated in Morphine Tolerant WT but not CB₂KO Mice with a History of LY2828360 Treatment

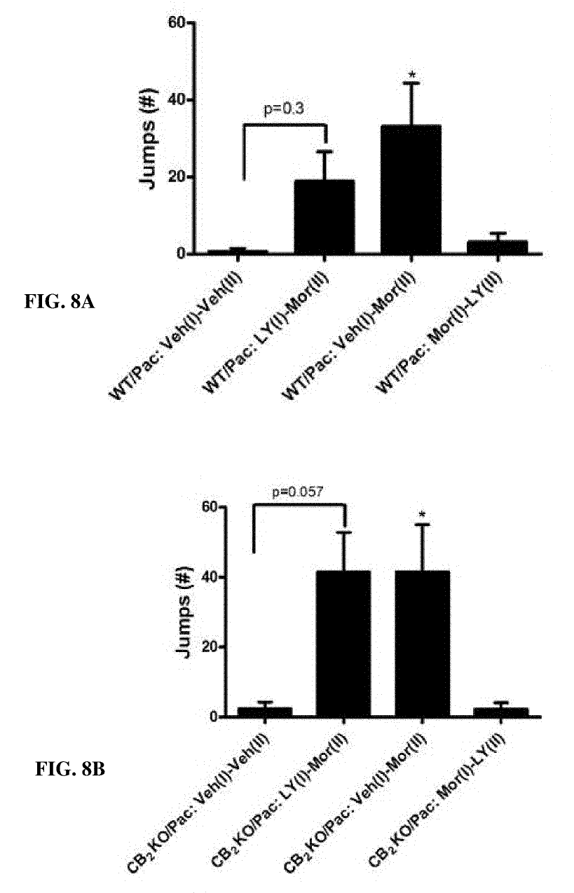

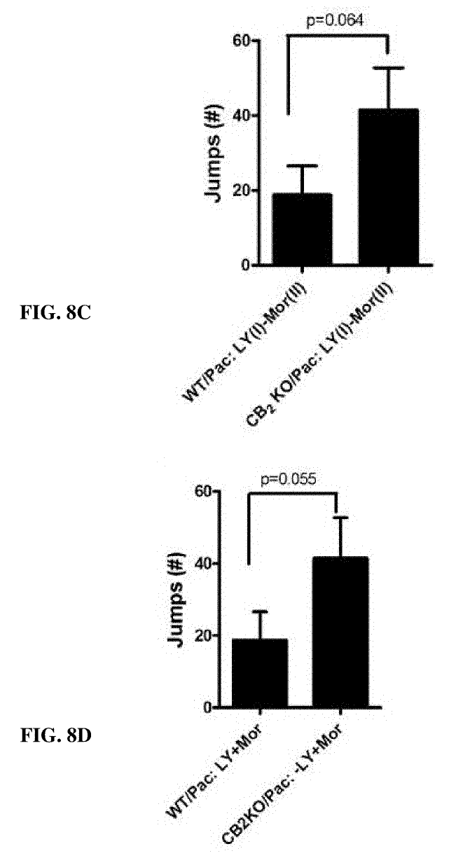

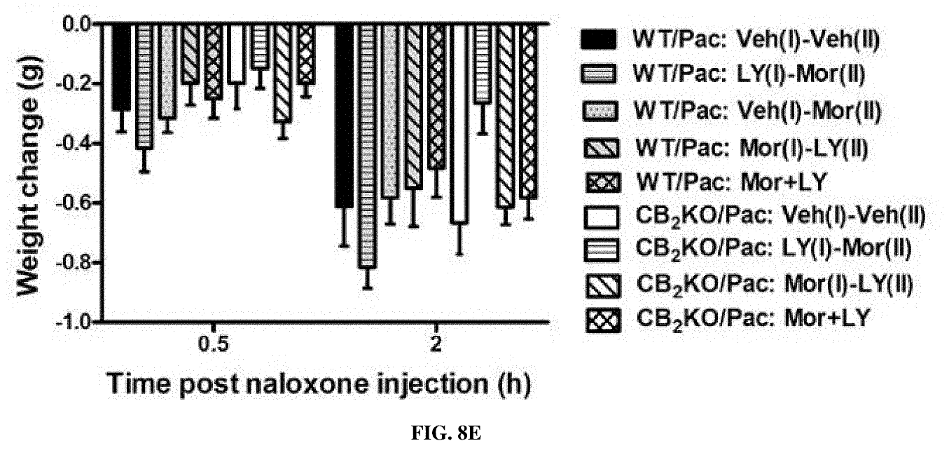

In paclitaxel-treated WT mice, a naloxone challenge produced characteristic jumping behavior that differed between groups (F_{3,22}=5.657, P=0.005) (FIG. 8A). Post hoc comparisons revealed that paclitaxel-treated WT mice that received morphine during phase 2 but vehicle during phase 1 [i.e., WT/Pac: Veh (1)-Mor (2) group] exhibited a greater number of jumps relative to paclitaxel-treated WT mice that received vehicle during both phases [WT/Pac: Veh (1)-Veh (2); P=0.007]. The number of naloxone-precipitated jumps did not differ between groups that received phase 1 LY2828360 followed by phase 2 morphine treatment [WT/Pac: LY (1)-Mor (2)] and those that received phase 1 vehicle followed by phase 2 vehicle treatment [WT/Pac: Veh (1)-Veh (2); P=0.3]. Also, the number of jumps did not differ between phase 2 morphine-treated mice that received either LY2828360 or vehicle during phase 1 [WT/Pac: Veh (1)-Mor (2) vs. WT/Pac: LY (1)-Mor (2), P=0.831]. In addition, the naloxone challenge did not precipitate withdrawal in paclitaxel-treated WT mice receiving morphine in phase 1 [WT/Pac: Mor (1)-LY (2) vs. WT/Pac: Veh (1)-Veh (2) P=1] (FIG. 8A).

Similarly, a naloxone challenge altered the number of jumps in paclitaxel-treated CB₂KO mice (F_{3,21}=5.696 P=0.005; FIG. 8B). In paclitaxel-treated CB₂KO mice, naloxone injection precipitated jumping in mice receiving phase 1 vehicle followed by phase 2 morphine treatment versus mice receiving vehicle during both phases of chronic dosing [CB₂KO/Pac: Veh (1)-Veh (2) vs. CB₂KO/Pac: Veh (1)-Mor (2), P=0.044].

The number of jumps trended higher in paclitaxel-treated CB₂KO mice receiving LY2828360 in phase 1 and morphine in phase 2 relative to CB₂KO mice that received vehicle during both phases [CB₂KO/Pac: LY (1)-Mor (2) vs. CB₂KO/Pac: veh (1)-Veh (2) group; P=0.057]. In paclitaxel-treated CB₂KO mice, the number of jumps did not differ between phase 2 morphine-treated mice that received either LY2828360 or vehicle during phase 1 [CB₂KO/Pac: LY (1)-Mor (2) vs. CB₂KO/Pac: Veh (1)-Mor (2), P=1]. A trend toward fewer naloxone-precipitated jumps was observed in WT relative to CB₂KO mice (P=0.064; FIG. 8C) that received the same histories of phase 1 LY2828360 followed by phase 2 morphine treatment.

Similarly, coadministration of LY2828360 with morphine also trended to produce a lower number of naloxone-precipitated jumps in WT compared with CB₂KO mice (P=0.055; FIG. 8D). The observed power of the marginally significant impaired t test comparing impact of LY2828360 on morphine-dependent WT and CB₂KO mice was 40%. A sample size of 20/group would be required to detect a statistically significant impact of LY2828360 on WT and CB₂KO animals based on the observed standard deviation (S.D.), sample size, and magnitude difference observed between means.

Body weight change from baseline (i.e., postvehicle) differed as a function of time after naloxone challenge (F_{1,48}=144.18, P=0.0001) but did not differ between groups. The interaction between time and group was not significant. A trend toward group differences in post-naloxone body weight was observed at 2 hours (F_{8,48}=2.033, P=0.062) but not at 0.5 hour (F_{8,48}=1.460, P=0.197) postinjection (FIG. 8E).

Impact of LY2828360 treatment on naloxone-precipitated opioid withdrawal in CB2KO and WT mice was observed. Naloxone (5 mg/kg i.p.) precipitates jumping in WT mice (FIG. 8A) and CB₂KO mice (FIG. 8B) receiving morphine (10 mg/kg per day i.p,×12 days) during phase 2 of chronic dosing. FIG. 8C shows a trend (P=0.064) toward lower numbers of naloxone-precipitated jumps was observed in WT compared with CB₂KO mice with similar histories of LY2828360 (3 mg/kg per day×10 days during phase 1), followed by morphine (10 mg/kg per day i.p.×12 days during phase 2) treatment.

FIG. 8D demonstrates naloxone-precipitated (5 mg/kg i.p.) jumping trended lower in WT mice (P=0.055) receiving coadministration of LY2828360 (0.1 mg/kg per day i.p.×12 days) with morphine (10 mg/kg per day i.p.×12 days) compared to CB₂KO mice with the same histories of drug treatment. Naloxone did not precipitate jumping behavior in the absence of morphine. Finally, FIG. 8E shows changes in body weight were greater at 2 hours compared with 0.5 hour after naloxone challenge. Data are expressed as mean±S.E.M. (n=6-8/group) *P<0.05 vs. Veh (I)-Veh (II), oneway analysis of variance followed by Bonferroni's post hoc test or one tailed t test as appreciate.

### Example 7: History of Chronic AM1710 Treatment Suppresses Paclitaxel-Induced Allodynia and Delays the Development of Tolerance to the Antiallodynic Effects of Morphine

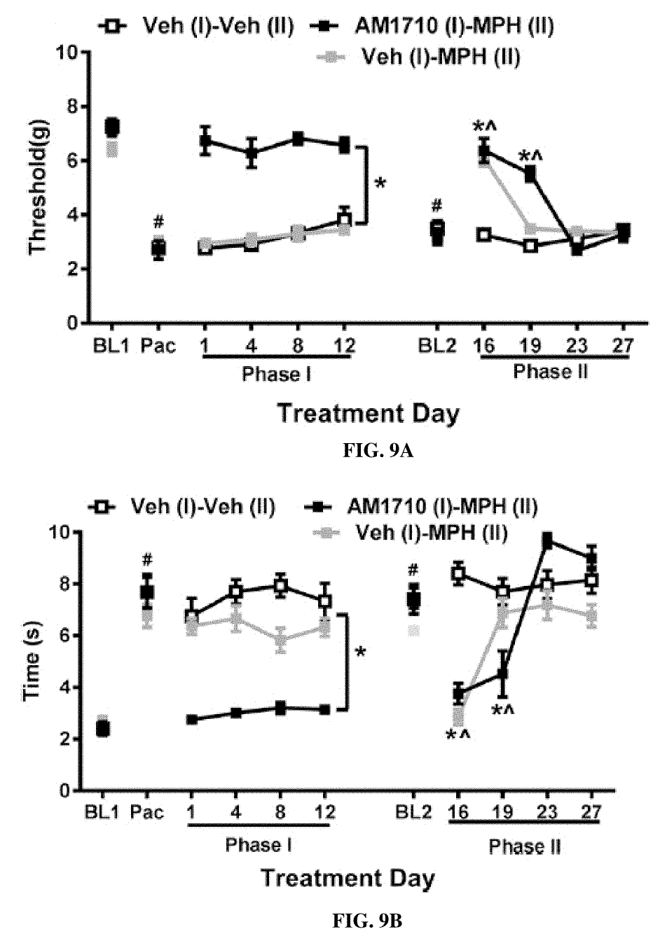

Paclitaxel (4 mg/kg, i.p.), administered on four alternate days, induced neuropathic pain in mice, as indicated by the reduction in the mechanical withdrawal threshold (F_{1,21}=544.316, P<0.001) (FIGS. 9A and 9BA) and increase in the response time to cold stimulation ((F_{1,21}=204.137, P<0.001) (FIGS. 9A and 9BB). No group difference was observed [F_{2,21}=0.644, P=0.535 (mechanical); F_{2,21}=0.284, P=0.755 (cold)] in mechanical or cold responsiveness before pharmacologic manipulations. An interaction between paclitaxel treatment and groups was detected for mechanical paw withdrawal threshold (F_{2,21}=4.463, P=0.024), although Bonferroni post hoc tests failed to detect any significant pairwise comparisons, suggesting that mechanical paw withdrawal thresholds did not differ between groups before phase I dosing. The interaction between chemotherapy treatment and groups for cold sensitivity was not significant (F_{2,21}=1.489, P=0.248). Thus, groups were similar before initiation of drug treatments.

To study the effects of AM1710 pretreatment on the development of tolerance to morphine, pharmacologic manipulations were used in two phases of treatment during the maintenance of neuropathic pain, when neuropathic pain was established and stable. AM1710 (5 mg/kg per day i.p.×12 days), administered once daily for 12 consecutive days to paclitaxel-treated WT mice during phase I, increased mechanical paw withdrawal thresholds (F_{2,21}=74.940, P<0.001) and reduced the heightened cold response time (F_{2,21}=52.339, P=0.001) compared with the vehicle treatment (FIGS. 9A and 9B). Mechanical and cold sensitivity returned to the baseline level measured before paclitaxel injection [P=0.521 (mechanical), P=0.374 (cold); planned comparison between baseline 1 and day 1 of phase I, paired t test]. The antiallodynic effect of AM1710 did not differ as a function of time [F_{6,63}=1.176, P−0.33 (mechanical); F_{6,63}=1.301, P=0.270 (cold)]. Mechanical paw withdrawal thresholds (F_{3,63}=3.329, P=0.025, Bonferroni post hoc test did not reveal any differences) and cold response times (F_{3,63}=1.189, P=0.321) remained stable throughout phase I treatment, indicating that tolerance did not develop to the antiallodynic effects of AM1710 over repeated administration for either stimulus modality (FIGS. 9A and 9B).

On day 15, 3 days after the completion of phase I of AM1710 treatment, mechanical and cold hypersensitivity returned to the level of hypersensitivity detected before AM1710 treatment [P=0.230 (mechanical), P=0.630 (cold); planned comparison between baseline 2 (BL2) and Pac in FIGS. 9A and 9B, paired t test). Chronic administration of morphine (10 mg/kg per day p.×12 days) was then initiated in phase II on day 16.

Overall, repeated morphine dosing in phase II reduced mechanical (F_{3,60}=53.59, P<0.001) and cold (F_{3,60}=32.45, P<0.001) responsiveness in paclitaxel-treated mice, but mechanical paw withdrawal thresholds (F_{2,20}=19.746, P<0.001) and cold response times (F_{6,60}=11.049, P=0.001) differed between groups. Mechanical and cold sensitivity in each group varied differently over repeated morphine administration (F_{6,60}=20.34, P<0.001 (mechanical); F_{6,60}=15.271, P<0.001 (cold)]. Specifically, morphine reduced mechanical (P<0.001) and cold (P<0.001) responsiveness in paclitaxel-treated mice relative to the vehicle group on the first day (day 16) of morphine treatment (FIGS. 9A and 9B).

By day 19, however, morphine was no longer efficacious in reducing paclitaxel-induced hypersensitivities in vehicle (I)-morphine (II)-treated groups, consistent with the development of morphine tolerance (FIGS. 9A and 9B). By contrast, morphine suppressed responsiveness to both modalities of cutaneous stimulation (P<0.001 mechanical; P=0.015 cold) on day 19 in paclitaxel-treated mice that received AM1710 (I)-morphine (II) treatment, although efficacy disappeared by day 23 (FIGS. 9A and 9B). These results indicate that a history of AM1710 treatment delayed the development of tolerance to morphine.

AM1710 sustainably suppressed paclitaxel-induced allodynia and delayed the development of morphine antinociceptive tolerance in mice. C57BL/J6 mice received a total of four doses of paclitaxel (4 mg/kg, i.p.) to develop peripheral neuropathic pain. After the paclitaxel-induced neuropathic pain was fully established, AM1710 (5 mg/kg per day×12 days) alone was administered during phase I, and 4 days after AM1710 administration, animals received chronic treatment of morphine (10 mg/kg per day×12 days) alone during phase II. AM1710 sustainably suppressed mechanical (FIG. 9A) and cold (FIG. 9B) allodynia induced by paclitaxel during phase I.

The history of AM1710 treatment during phase I delayed the development of morphine tolerance in phase II shown in FIGS. 9A and 9B (n=8 males, C57BL/6J for each group. *P<0.05 vs. BL (baseline); *P<0.05 vs. veh (I)-veh (II); ^{A}P<0.05 vs. day 23 (two-way mixed ANOVA, followed by Bonferroni post hoc test)). BL, baseline; MPH, morphine; veh, vehicle.

### Example 8: Naloxone-Precipitated Opioid Withdrawal was Decreased in Morphine-Tolerant Mice with a History of AM1710 Treatment

We also evaluated whether prior chronic treatment with AM1710 (5 mg/kg i.p.×12 days) in phase I would impact naloxone-precipitated morphine withdrawal symptoms in mice rendered tolerant to morphine (10 mg/kg i.p.×12 days) in phase II. The number of naloxone-precipitated jumps differed reliably between groups (F_{2,19}=7.264, P=0.0045; one-way ANOVA). Paclitaxel-treated mice that received vehicle (I)-morphine (II) treatment exhibited a greater number of jumps compared with vehicle (I)-vehicle (II)-treated mice that never received morphine (P=0.002; Bonferroni post hoc test) (FIG. 10A). Moreover, naloxone-precipitated jumps did not differ between the AM1710 pretreatment [i.e., AM1710 (I)-morphine (II)] and vehicle [i.e., vehicle (I)-vehicle (II)) groups (P=0.188; Bonferroni post hoc test] (FIG. 10A). The number of naloxone-precipitated jumps was lower in the AM1710 (I)-morphine (II)) group compared with the vehicle (I)-morphine (II) group that received identical morphine treatments (P=0.042; Bonferroni multiple comparison test).

These observations suggest that AM1710 attenuated naloxone-precipitated withdrawal jumps in morphine-dependent mice, and that withdrawal jumping was normalized by AM1710 pretreatment. AM1710 did not alter the effects of naloxone challenge on body weight or body temperature. Body weight decreased over time after naloxone injection (F_{1,19}=36.052, P<0.001), which was independent of the treatment (F_{2,19}=0.626, P=0.546), and weight loss did not differ among treatments (F_{2,19}=0.219, P=0.806; FIG. 10B). Similarly, no differences were observed between treatments with respect to changes in body temperature induced by naloxone challenge (F_{2,21}=1.390, P=0.273; FIG. 10C).

AM1710 attenuates naloxone-precipitated opioid withdrawal. Paclitaxel-treated mice rendered tolerant to morphine were challenged with naloxone (5 mg/kg, i.p.) to induce physical withdrawal. Animals pretreated with AM1710 (5 mg/kg per day×12 days, i.p.) before morphine (MPH) treatment (10 mg/kg, i.p.) for 12 days exhibited less jumping behavior compared with animals receiving morphine alone (FIG. 10A). Weight loss did not differ among treatments (FIG. 10B). Body temperature changes did not differ among treatments (FIG. 10C).

The history of AM1710 treatment during phase I delayed the development of morphine tolerance in phase II shown in FIGS. 10A-C (n=8 males, C57BL/6J for each group, <0.01 vs. vehicle (I)-vehicle (II) (one-way ANOVA, followed by Bonferroni post hoc test); ^{#}P<0.05 vs. vehicle (I)-morphine (II) (one-way ANOVA, followed by Bonferroni multiple comparison test). ^{AAA} P<0.001 vs. post 30 minutes (two-way mixed ANOVA) veh, vehicle; MPH, morphine.

### Example 9: LY2828360 Displays a Delayed, G Protein-Biased Signaling Profile at Human CB₂ Receptors

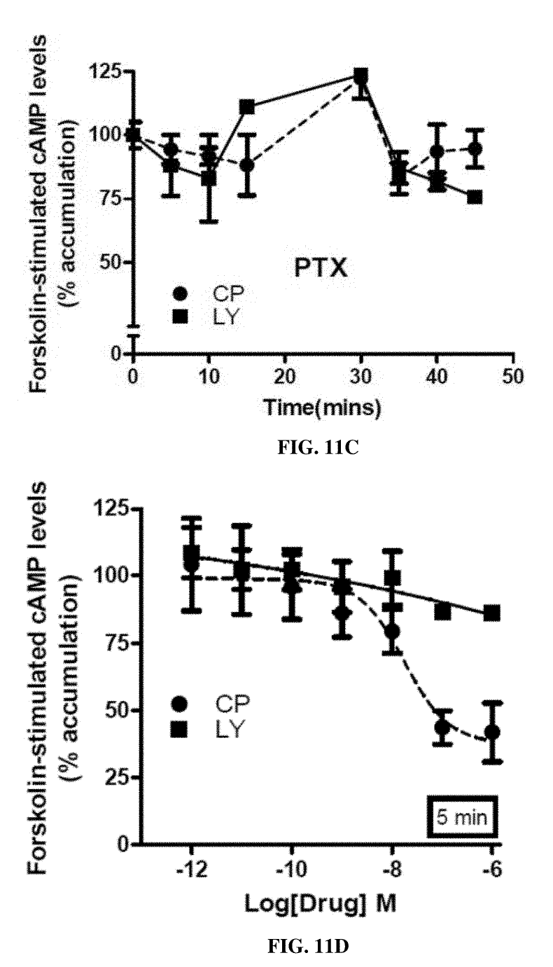

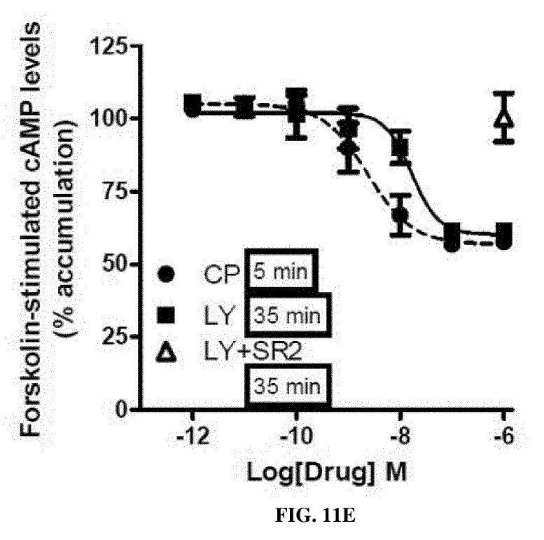

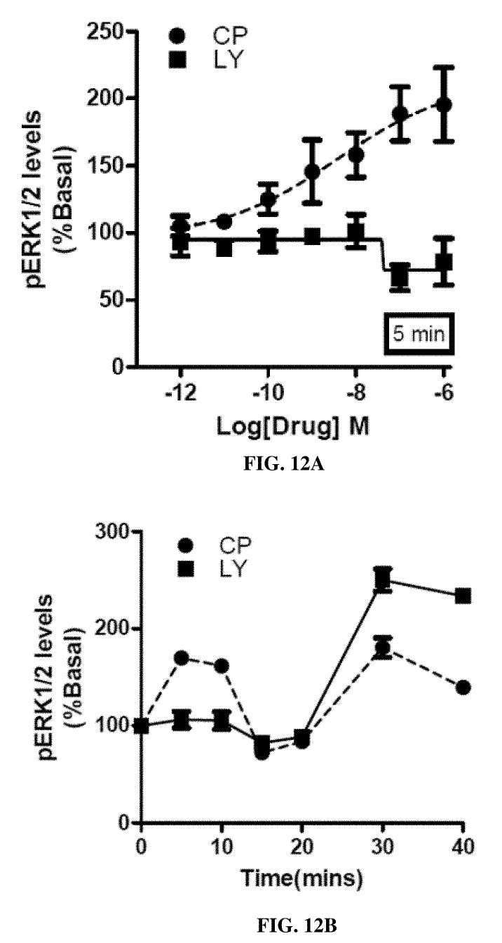

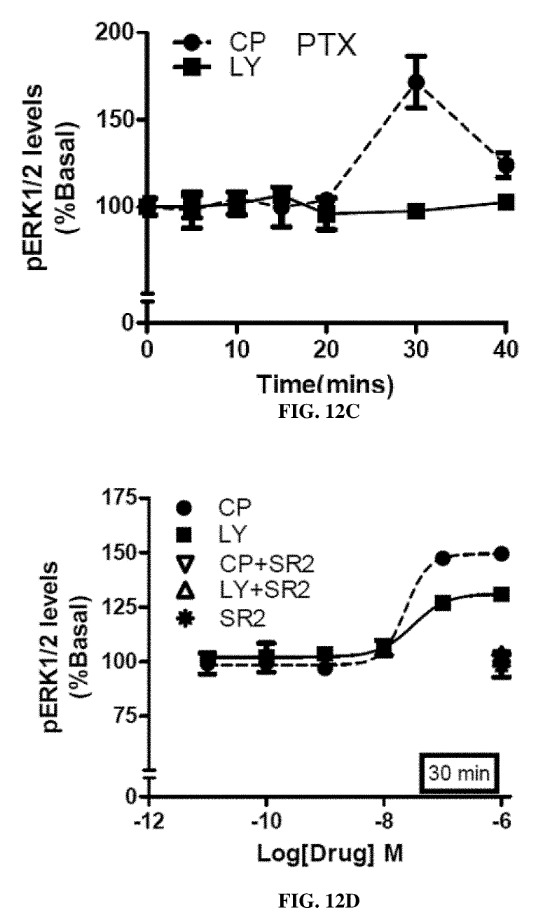

To determine whether the slow, biased signaling of LY2828360 was specific for mouse CB₂ receptors (mCB2), LY2828360 signaling via human CB₂ (hCB2) receptors was evaluated. As with mCB₂, LY2828360 failed to internalize hCB2 receptors (FIG. 11A). The LY2828360 compositions also exhibited time-dependent delayed inhibition of cAMP accumulation (FIGS. 11B, 11D, and 11E) and ERK1/2 phosphorylation (FIGS. 12A, 12B, and 12D). As with mouse CB₂ receptors, these effects observed with the LY2828360 composition in human were abolished by pertussis toxin (PTX) treatment (FIGS. 11C and 12C) and blocked by SR144528 (FIGS. 11E and 12D), confirming the involvement of G_{i/o} proteins and CB₂ receptors, respectively.

LY2828360 displays a delayed signaling profile at human CB2 receptors. In HEK cells stably expressing human CB2 receptors, LY2828360 failed to internalize the receptor (FIG. 11A). In a forskolin-stimulated cAMP time course assay, CP55940 (1 pM) inhibited cAMP accumulation at 5 minutes, while LY2828360 (1 pM) displayed a similar efficacy only after 35 minutes of agonist incubation (FIG. 11B). Pertussis toxin (PTX) pretreatment abolished this effect at all the time points tested and/or examined for both drugs (FIG. 11C). After 5 minutes of treatment, CP55940 was potent and efficacious in inhibiting forskolin-stimulated cAMP accumulation while LY2828360 had no effect (FIG. 11D). After 35 minutes, LY2828360 was a potent and efficacious agonist in inhibiting forskolin-stimulated cAMP accumulation and this inhibition was completely blocked by a CB₂ receptor antagonist, SR144528 (FIG. 11E).

In the pERK 1/2 assay, CP55940, at the 5 min time point, was potent and efficacious in increasing ERK1/2 phosphorylation, while LY2828360 was ineffective (FIG. 12A). Examination of a time course of ERK1/2 phosphorylation revealed that LY2828360 (1 pM) increased ERK1/2 phosphorylation after 30 minutes, but not at 5 minutes (FIG. 12B). In contrast, CP55940 (1 pM) efficaciously increased ERK1/2 phosphorylation after 5 minutes, 10 minutes, and 30 minutes (FIG. 12B). Pertussis toxin (PTX) treatment abolished ERK1/2 phosphorylation after treatment with LY2828360 (1 pM), while CP55940-stimulated phosphorylation of pERK 1/2 after 30 minutes was retained (FIG. 12C). LY2828360- and CP55940-stimulated phosphorylation of ERK1/2 was completely blocked by SR144528 (1 pM) (SR2; FIG. 12D).

Forskolin-stimulated cAMP assays were performed in duplicates. All other assays were performed in triplicates. All experimental data were plotted and analyzed using GraphPad Prism 4.

### Example 10: LY2828360 Effect on IP1 Accumulation Via CB₂ Receptors

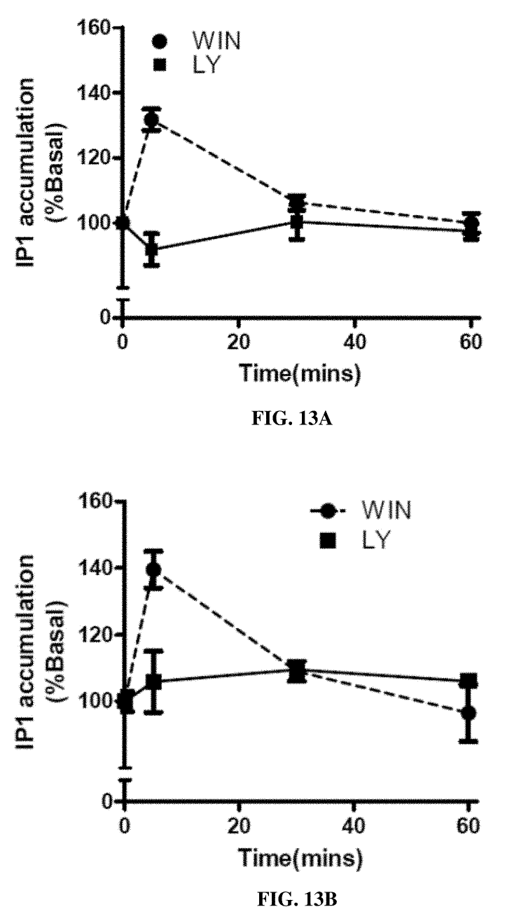

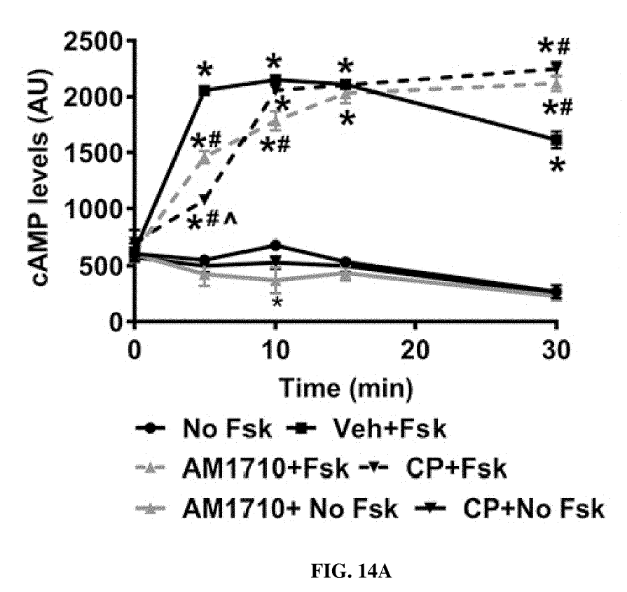

Finally, LY2828360 did not affect IP1 accumulation via mouse CB₂ (FIG. 13A) or human CB₂ receptors (FIG. 13B). Moreover, LY2828360 failed to affect IP1 levels through either mouse or human CB₂ receptors (FIGS. 13A and 13B). WIN55212-2 increased IP1 accumulation after 10 minutes by either mouse or human CB₂ receptors.

Assays were performed using HEK cells stably expressing mouse or human CB₂ receptors. IP1 assays were performed in triplicates and the data were plotted and analyzed using GraphPad Prism 4.

Here, the CB₂ agonist LY2828360 is a slow-acting but efficacious G protein-biased CB₂ agonist that inhibits cAMP accumulation and activates ERK1/2 signaling in vitro. In vivo, chronic systemic administration of the CB₂ agonist LY2828360 suppressed chemotherapy-induced neuropathic pain without producing tolerance. The observed anti-allodynic efficacy was absent in CB₂KO mice, demonstrating mediation by CB₂ receptors.

Sustained efficacy of LY2828360 was observed in mice with a history of morphine tolerance. Moreover, both chronic LY2828360 dosing completed before morphine dosing and coadministration of LY2828360 with morphine strongly attenuated development of tolerance of morphine. LY2828360 also trended to decrease naloxone precipitated withdrawal signs in WT but not in CB₂KO mice.

LY2828360 also displays an intriguing, yet interesting, signaling profile at mouse and human CB₂ receptors. These results indicate that LY2828360 is a slow-acting CB₂-receptor agonist strongly biased toward G_{i/o}G protein signaling with little effect on arrestin or G_{q} signaling, which contrasts strongly with the balanced agonist CP55940 and AM710, which rapidly inhibited cAMP accumulation and increased pERK1/2. Therefore, the AM710 is representative of a functionally balanced, fast-acting compound of the present methods which has efficacy in treating pain, opioid tolerance, and opioid withdrawal. However, the ability of a ligand to selectively activate a subset of signaling pathways, as demonstrated herein by LY2828360, is termed “biased agonism” or “functional selectivity,” and has emerged as an important pharmacologic mechanism providing an advantageous pharmaceutical effect and/or outcome for the patient, which was unexpected.

For example, a “biased” agonist may activate a pathway that is therapeutically more relevant and shun pathways that lead to untoward effects. More recently, “kinetic bias” has emerged as another important pharmacologic mechanism that emphasizes the time scale of the activation of a particular pathway, particularly the slow activation (i.e., at about, at least, or not less than about 30 minutes for activation) that has been observed for the LY2828360 compound. It remains to be determined whether the marked kinetic and G-protein bias of LY2828360 explains either its remarkable opioid sparing property or its failure in clinical trials for osteoarthritis pain.

Tolerance limits therapeutic utility of an analgesic. In the present study, the antiallodynic efficacy of LY2828360 was fully maintained in neuropathic subjects, such as mice, that received once daily administration of the maximally effective dose of LY2828360 over 12 consecutive days. Antiallodynic efficacy of LY2828360 (3 mg/kg i.p.) lasted more than 4.5 hours after acute administration. Responsiveness to mechanical and cold stimulation returned to baseline after 1 and 3 days, respectively. Our data also supports and are consistent with studies showing that CB₂ agonist AM1710 suppresses paclitaxel-induced neuropathic pain without producing tolerance or physical dependence after either 8 days of once daily (i.p.) dosing or chronic infusion over 4 weeks.

A striking observation of the present study was that prior chronic treatment with LY2828360 for 12 days prevented subsequent development of tolerance to the antiallodynic effect of morphine. By contrast, tolerance to morphine developed in CB₂KO mice identically treated with chronic LY2828360 in phase 1 followed by chronic morphine treatment in phase 2. Moreover, in paclitaxel-treated WT mice, coadministration of morphine with a low dose of LY2828360 was fully efficacious in alleviating neuropathic pain and blocking the development of morphine tolerance. These observations suggest that analgesic efficacy and, potentially, the therapeutic ratio of morphine could be improved by adjunctive treatment that combines an opioid with a CB₂ agonist to treat neuropathic pain while simultaneously limiting the development of tolerance, dependence, and potentially other adverse side effects of the opioid analgesic.

Our results are in line with a recent report that coadministration of a low dose of the CB₂ receptor agonist AM1241 combined with morphine reduced the morphine tolerance in Walker 256 tumor-bearing rats, although mediation by CB₂ receptors was not assessed. AM1241 produced a modest enhancement of opioid-mediated antinociception in the hotplate test and in a test of mechanical sensitivity in tumor-bearing rats. However, tolerance developed to the antiallodynic effects of the combination treatment assessed with mechanical but not thermal (hot plate) stimulation, suggesting that therapeutic benefit of the adjunctive treatment may be ligand- and/or modality-dependent. Coadministration of CB₂ agonist JWH133 also exhibited opioid-sparing effects in the formalin model of inflammatory pain. The mechanism underlying these therapeutically advantageous properties remains incompletely understood.

In tumor-bearing mice, AM1241 upregulated μ-opioid receptor expression in the spinal cord and dorsal root ganglia (DRG). Another study suggested CB₂ agonist upregulated μ-opioid receptor expression levels, whereas the CB₂ antagonist inhibited μ-opioid receptor expression level in Jurkat T cells and in mouse brainstem. Mitogen-activated protein kinase (MAPK) activation and glial proinflammatory mediator release have also been linked to morphine tolerance. CB₂ agonists could alleviate morphine tolerance by an interaction between microglial opioid and CB₂ receptors and/or by reduction of glial and MAPK activation.

CB₂ activation is correlated with increasing anti-inflammatory gene expression in the dorsal horn and reductions in mechanical and thermal hypersensitivities. Coadministration of morphine with the CB₂ agonist JWH015 synergistically inhibited preclinical inflammatory, postoperative, and neuropathic pain in a dose- and time-dependent manner. The observed synergism may involve activation of CB₂ receptors on immune cells and subsequent inhibition of the inflammatory process coupled with morphine's well characterized ability to inhibit nociceptive signaling.

In keratinocytes in peripheral paw tissue, AM1241 stimulated the release of the endogenous opioid β-endorphin, which acted at local neuronal MORs to inhibit nociception through a naloxone-dependent mechanism; however, naloxone sensitivity is not a class effect of CB₂ agonists and cannot account for AM1241 antinociception but may depend upon levels of endogenous analgesic tone.

Some effects of cannabinoid receptor agonists and antagonists on morphine antinociceptive tolerance remain controversial. Coadministration of the CB₂ receptor agonist JWH015 with morphine increased morphine analgesia and morphine antinociceptive tolerance. By contrast, the CB₂ receptor antagonist JTE907 decreased morphine analgesia and attenuated morphine antinociceptive tolerance in rats using tail-flick and hot-plate tests of antinociception. Differences in experimental paradigms, biased signaling of the CB₂ agonist used, or the presence or absence of a pathologic pain state could account for these disparities.

An emerging challenge for pain management is how to treat pain in the morphine-tolerant individual. Dose escalation is typically used in early unimodal treatment, which may enhance potential for abuse. The combination of two or more analgesic agents with different mechanisms was proposed as an analgesic strategy. Our study has important implications for the clinical management of neuropathic pain because chronic LY2828360 and AM1710 treatment showed sustained antiallodynic efficacy in neuropathic mice previously rendered tolerant to morphine. This observation is unlikely to be due to pharmacokinetic factors because morphine dosing ceased for 4 days in our study before introduction of phase 2 LY2828360 chronic treatment.

Physical dependence is another major side effect of opioid treatment, which can lead to a withdrawal syndrome when the user stops taking the drug; however, most studies of opioid dependence have used naive animals rather than animals subjected to a neuropathic pain state. The opioid receptor antagonist naloxone precipitates a spectrum of autonomic and somatic withdrawal signs in morphine-dependent animals. In the present study, in paclitaxel-treated WT mice, chronic phase 1 pretreatment with LY2828360 produced a trend toward reducing naloxone-precipitated a plurality of withdrawal jumps without reducing pain relief in the same animals where LY2828360 blocked development of morphine tolerance.

This trend was absent in CB₂KO mice receiving identical treatments. In fact, our studies raise the possibility that CB₂ receptor signaling may attenuate opioid antagonist-precipitated withdrawal because CB₂KO mice trended to show higher levels of naloxone-precipitated jumping compared with WT mice when pretreated with CB₂ agonist. Moreover, coadministration of low-dose LY2828360 with morphine mimicked these effects and trended to decrease naloxone-precipitated withdrawal jumping in paclitaxel-treated WT mice compared with CB₂KO mice (P=0.055).

Thus, LY2828360 may be efficacious in decreasing morphine withdrawal symptoms, such as a plurality of withdrawal jumps. Variability in withdrawal jumps and inadequate statistical power could account for the failure to observe more robust statistical differences in jumps between groups; the primary endpoints evaluated here were mechanical and cold responsiveness, not naloxone-induced jumping. Observations from both these studies are, nonetheless, broadly consistent with the hypothesis that CB₂ receptor activation may attenuate signs of opioid withdrawal. Stimulation of microglial CB₂ receptors by the CB₂ agonist suppressed microglial activation, which has been linked to morphine withdrawal behaviors. Thus, depletion of spinal lumbar microglia decreased withdrawal behaviors and attenuated the severity of withdrawal without affecting morphine antinociception. The mechanism underlying these observations remains to be explored.

In summary, our observations suggest that CB₂ agonists may be useful as a first-line treatment of suppressing chemotherapy-induced neuropathic pain with tolerance (e.g., AM1710) or without tolerance (e.g., LY2828360). In particular, these results suggest that CB₂ agonists, particularly LY2828360, may be useful for suppressing neuropathic pain with sustained efficacy in opioid-recalcitrant pain states without the development of tolerance or dependence.

Accordingly, the methods described herein comprise, consist essentially of, or consist of administration of active compositions such as LY2828360 or AM1710, to subjects in order to suppress, reduce, prevent, or delay neuropathic pain without tolerance or dependence. In addition, the methods of the present disclosure are related to administration of compositions, such as LY2828360 or AM1710, to subjects to suppress or delay opioid tolerance, respectively. Further, the methods of the present disclosure are related to administration of compositions, such as LY2828360 or AM1710, to subjects to suppress, delay, or prevent opioid withdrawals (e.g., withdrawal jumps).

Various modification and variation of the described methods and compositions of the present application will be apparent to those skilled in the art without departing from the scope and spirit of the present application. Although the present application has been described in connection with specific preferred embodiments, it should be understood that the present application as claimed should not be unduly limited to such specific embodiments. Indeed, various modifications of the described modes for carrying out the present application that are obvious to those skilled in the relevant fields are intended to be within the scope of the following claims.

### Example 11: AM1710 Inhibited Forskolin-Stimulated cAMP Accumulation in HEK Cells Expressing mCB2 or hCB2, but the Kinetics of Inhibition Differed Between mCB2 and hCB2

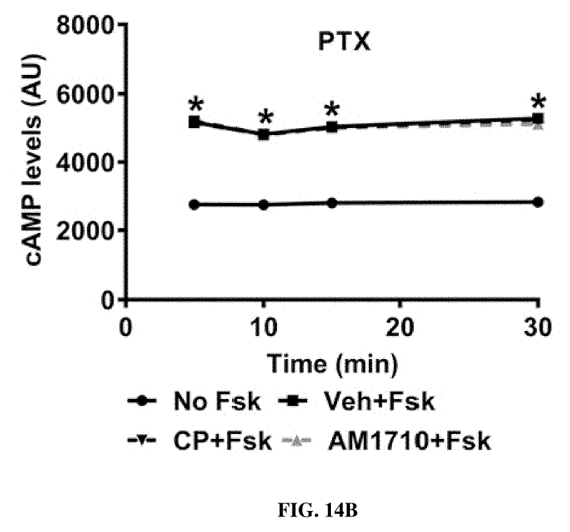

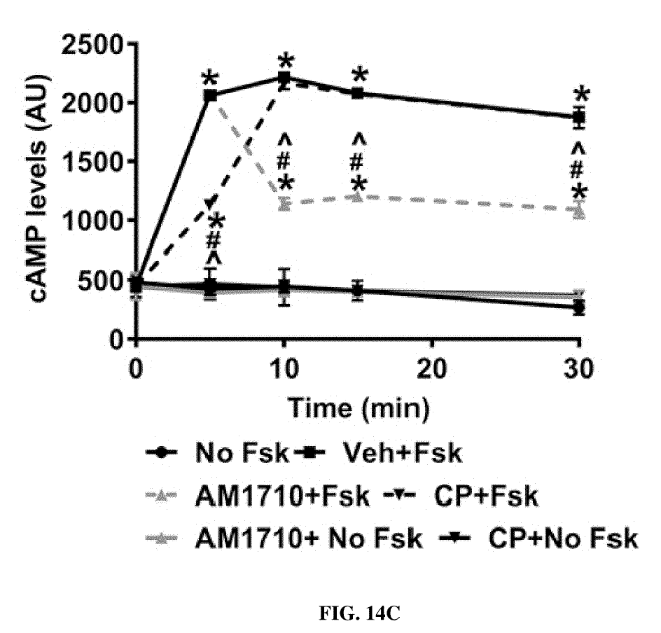

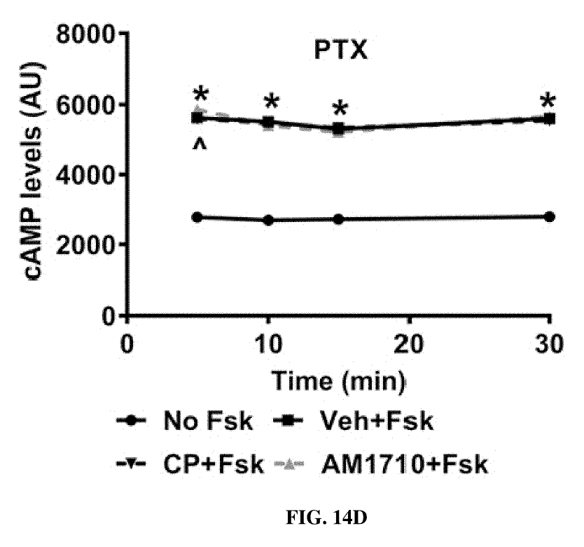

In HEK cells stably expressing mCB2, cAMP levels differed between treatments (F_{5,12}=609, P<0.001) and varied over time (F_{4,48}=108.2, P<0.001) (FIG. 14A). The interaction between treatment and time was significant (F_{20,48}=44.58, P<0.001) (FIG. 14A). Forskolin persistently increased cAMP levels in cells incubated with vehicle, starting at 5 minutes (P<0.001). The presence of CP55940 (1 μM final concentration) (P<0.001) or AM1710 (1 μM final concentration) (P<0.001) attenuated forskolin-induced cAMP levels at 5 minutes.

Although CP55940 exhibited a stronger inhibitory effect than AM1710 at 5 minutes (P<0.001), the inhibitory effect of AM1710 outlasted that of CP55940, and the inhibition induced by AM1710 dissipated by 15 minutes. After the brief inhibition of cAMP levels by CP55940 or AM1710, cAMP levels exceeded those in cells treated with forskolin alone (P<0.001) (FIG. 14A). In the absence of forskolin, CP55940 or AM1710 alone did not change cAMP levels, as no differences were observed between these conditions and the basal/no forskolin condition with one exception; AM1710 treatment alone decreased cAMP levels below the basal/no forskolin level at 10 minutes (P=0.012). Pertussis toxin (PTX) pretreatment abolished the decrease in forskolin-stimulated cAMP induced by either CP55940 (1 μM final concentration) or AM1710 (1 μM final concentration) in HEK cells stably expressing mCB2 (FIG. 14B). Despite the significant changes in cAMP over time (F_{3,24}=29.51, P<0.001) and significant effects of both treatment (F_{3,8}=1443, P<0.001) and interaction (F_{9,24}=2.795, P=0.021), no differences were detected between treatments with forskolin stimulation at any time point in the PTX-treated cells (P>0.235) (FIG. 14B).

In HEK cells stably expressing hCB2, cAMP levels differed between treatments (F_{5,12}=412.6, P<0.001) and varied over time (F_{4,48}=123.9, P<0.001) (FIG. 14C). The interaction between treatment and time was significant (F_{20,48}=47.54, P<0.001) (FIG. 14C). Similarly, forskolin persistently increased cAMP levels in cells incubated with vehicle starting at 5 minutes (P<0.001); however, only CP55940 produced early inhibition of forskolin-induced cAMP levels at 5 minutes (P<0.001) (FIG. 14C).

By contrast, AM1710 induced a delayed and persistent inhibition of forskolin-stimulated cAMP levels, starting at 10 minutes (P<0.001) (FIG. 14C). Like the HEK cells stably expressing mCB2, CP55940 and AM1710 alone did not change the cAMP levels in cells stably expressing hCB2 (P=1). PTX pretreatment blocked the inhibition of forskolin-stimulated cAMP produced by either CP55940 (1 μM final concentration) or AM1710 (1 μM final concentration) in HEK cells stably expressing hCB2 (FIG. 14D). Despite the significant changes over time (F_{3,24}=22.95, P<0.001) and significant effects of both treatment (F_{3,8}=2472, P<0.001) and interaction (F_{9,24}=3.391, P=0.008), no differences were detected between treatments with forskolin stimulation at any time point (P>0.831), with one exception: in the presence of forskolin, AM1710 increased cAMP levels compared with forskolin alone at 5 minutes in PTX-treated cells (P=0.048) (FIG. 14D).

AM1710 inhibited forskolin-stimulated cAMP in HEK cells expressing mCB2 and hCB2, but the kinetics of inhibition differed between mCB2 and hCB2. In HEK cells expressing mCB2, both CP55940 and AM1710 reduced cAMP levels at 5 minutes (FIG. 14A). The inhibitory effect of AM1710 lasted longer than CP55940 and dissipated by 15 minutes. After treating HEK cells expressing mCB2 with PTX, both CP55940 and AM 1710 failed to reduce cAMP levels at all time points examined (FIG. 14B). In HEK cells expressing hCB2, CP55940 induced early reduction of cAMP at 5 minutes, which lasted up to 10 minutes, whereas AM 1710 induced a delayed (at 10 minutes) but long-lasting (up to 30 minutes) decrease in cAMP (FIG. 14C). After treating HEK cells expressing hCB2 with PTX, both CP55940 and AM 1710 failed to reduce cyclase levels at all time points examined (FIG. 14D).

*P<0.05 vs. No Fsk; *P<0.05 vs. Veh+Fsk, ^{A}P<0.05 significant difference between CP+Fsk and AM1710+Fsk (two-way mixed ANOVA, followed by Bonferroni' post hoc test). AU, arbitrary unit; CP, CP55940; Fsk, forskolin; hCB2, human CB2 receptors; mCB2, mouse CB2 receptors; Veh, vehicle, n=3 for each group.

What is claimed is:

1. A method of suppressing neuropathic pain without producing tolerance in a subject, the method comprising:
a) administering a pharmaceutical composition comprising a cannabinoid CB2 receptor agonist compound to the subject,
b) activating one or more G-protein signaling pathways that is sufficient to affect neuropathic pain in the subject,
c) improving one or more clinical manifestations of the neuropathic pain in the subject, and
d) suppressing the neuropathic pain in the subject.

2. The method of claim 1, wherein the subject is a human or a rodent.

3. The method of claim 1, wherein the cannabinoid CB2 receptor agonist compound comprises a LY2828360 compound or an analog, a derivative, a pharmaceutically acceptable salt, a hydrate, a prodrug, or a combination thereof.

4. The method of claim 1, wherein the cannabinoid CB2 receptor agonist compound comprises an AM1710 compound or an analog, a derivative, a pharmaceutically acceptable salt, a hydrate, a prodrug, or a combination thereof.

5. The method of claim 3, wherein the LY2828360 compound comprises (8-(2-chlorophenyl)-2-methyl-6-(4-methylpiperazin-1-yl)-9-(tetrahydro-2H-pyran-4-yl)-9H-purine).

6. The method of claim 3, wherein the LY2828360 compound has the following chemical structure:

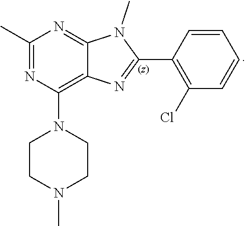 [SMILES: Cc1nc(N2CCN(C)CC2)c2nc(-c3ccccc3Cl)n(C)c2n1]

7. The method of claim 4, wherein the AM1710 compound comprises 3-(1,1-dimethyl-heptyl)-1-hydroxy-9-methoxy-benzo(c) chromen-6-one or has the following chemical structure:

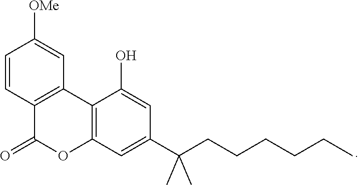 [SMILES: CCCCCCC(C)(C)c1cc(O)c2c(c1)oc(=O)c1ccc(OC)cc12]

8. The method of claim 3, wherein activating the one or more G-protein signaling pathways by the LY2828360 compound occurs through a slow signaling mechanism.

9. The method of claim 4, wherein activating of the one or more G-protein signaling pathways by the AM1710 compound occurs through a fast signaling mechanism.

10. A method of reducing or preventing opioid withdrawal in a subject, the method comprising:
a) administering to the subject a pharmaceutical composition comprising a LY2828360 compound, an AM1710 compound, or an analog, a derivative, a pharmaceutically acceptable salt, a hydrate, a prodrug, or a combination thereof,
b) activating one or more G-protein signaling pathways that affects opioid withdrawal in the subject,
c) suppressing the one or more clinical manifestations of the opioid withdrawal in the subject, and
d) reducing or preventing opioid withdrawal in the subject.

11. The method of claim 10, wherein the LY2828360 compound comprises (8-(2-chlorophenyl)-2-methyl-6-(4-methylpiperazin-1-yl)-9-(tetrahydro-2H-pyran-4-yl)-9H-purine).

12. The method of claim 10, wherein the LY2828360 compound has the following chemical structure:

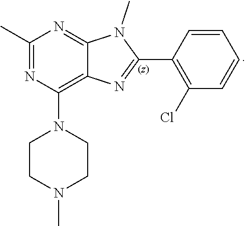 [SMILES: Cc1nc(N2CCN(C)CC2)c2nc(-c3ccccc3Cl)n(C)c2n1]

13. The method of claim 10, wherein the AM1710 compound comprises 3-(1,1-dimethyl-heptyl)-1-hydroxy-9-methoxy-benzo(c) chromen-6-one or has the following chemical structure:

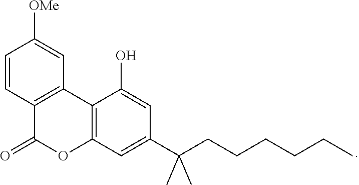 [SMILES: CCCCCCC(C)(C)c1cc(O)c2c(c1)oc(=O)c1ccc(OC)cc12]

14. The method of claim 10, wherein the one or more clinical manifestations of the opioid withdrawal comprises a plurality of withdrawal jumps.

15. The method of claim 10, wherein the subject is a human or a rodent.

16. A method of reducing or preventing the development of opioid tolerance in a subject, the method comprising:
a) co-administering to the subject one or more pharmaceutical compositions
comprising;
i) a LY2828360 compound, an AM1710 compound, or an analog, a derivative, a pharmaceutically acceptable salt, a hydrate, a prodrug, or a combination thereof, and
ii) an opioid;
b) activating one or more G-protein signaling pathways that effects opioid tolerance in the subject,
c) suppressing the development or presentation of one or more clinical manifestations of the opioid tolerance in the subject, and
d) reducing or preventing the development of opioid tolerance in the subject.

17. The method of claim 16, wherein the subject is a human or a rodent.

18. The method of claim 16, wherein the opioid is selected from the group consisting of morphine, codeine, oxycodone, oxycontin, hydrocodone, methadone, meperidine, buprenorphine, hydromorphone, tapentadol, tramadol, heroin, fentanyl, and their anlaogs.

19. The method of claim 16, wherein the LY2828360 compound comprises (8-(2-chlorophenyl)-2-methyl-6-(4-methylpiperazin-1-yl)-9-(tetrahydro-2H-pyran-4-yl)-9H-purine) or the AM1710 compound comprises 3-(1,1-dimethyl-heptyl)-1-hydroxy-9-methoxy-benzo(c) chromen-6-one.

20. The method of claim 16, wherein the LY2828360 compound has the following chemical structure: 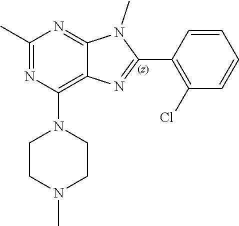 [SMILES: Cc1nc(N2CCN(C)CC2)c2nc(-c3ccccc3Cl)n(C)c2n1]
or the AM1710 compound has the following chemical structure: 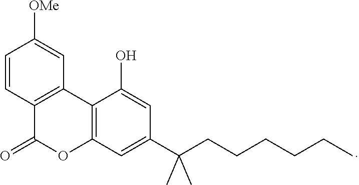 [SMILES: CCCCCCC(C)(C)c1cc(O)c2c(c1)oc(=O)c1ccc(OC)cc12]
